(19) United States
(12) Patent Application Publication
(10) Pub. No.: US 2025/0036036 A1
(43) Pub. Date: Jan. 30, 2025

(54) ELECTROPHOTOGRAPHIC PHOTOCONDUCTOR, ELECTROPHOTOGRAPHIC PHOTOCONDUCTOR CARTRIDGE, IMAGE FORMATION DEVICE, AND COMPOUND

(71) Applicant: MITSUBISHI CHEMICAL CORPORATION, Tokyo (JP)

(72) Inventors: Tsukasa HASEGAWA, Tokyo (JP); Manuel Emilio OTERO RAMIREZ, Tokyo (JP); Akira ANDO, Tokyo (JP); Hideki GOROUMARU, Tokyo (JP)

(73) Assignee: MITSUBISHI CHEMICAL CORPORATION, Tokyo (JP)

(21) Appl. No.: 18/901,884

(22) Filed: Sep. 30, 2024

Related U.S. Application Data
(63) Continuation of application No. PCT/JP2023/012826, filed on Mar. 29, 2023.

(30) Foreign Application Priority Data
Mar. 30, 2022 (JP) 2022-055574
Mar. 30, 2022 (JP) 2022-055575

(51) Int. Cl. G03G 5/147

(52) U.S. Cl. CPC G03G 5/14713; G03G 2215/00957; G03G 2215/00962

(57) ABSTRACT

The present invention provides an electrophotographic photoconductor having a photosensitive layer and a protective layer in sequence on a conductive support, the protective layer containing an electron transporting compound having a left-right asymmetric structure in which an electron transporting skeleton is centered, with a side chain having a polymerizable functional group on one side, and with a side chain having no polymerizable functional group on the other side, the electrophotographic photoconductor having good electrical properties, especially residual potential properties, even when a compound having electron transportability is contained in the protective layer.

### TECHNICAL FIELD

The present invention relates to a compound having electron transportability, such as a compound useful as an electron transporting compound used as a raw material for an electrophotographic photoconductor used in copiers, printers, and the like, an electrophotographic photoconductor used in copiers, printers, and the like, and an electrophotographic photoconductor cartridge and an image formation device using the same.

### BACKGROUND ART

In printers, copiers, and the like, a charged organic photoconductor (OPC) drum is irradiated with light to form an electrostatic latent image by removing the electricity from the irradiated portion, and a toner adheres to the electrostatic latent image to provide an image. In devices using such electrophotographic technology, the photoconductor is a key member.

The organic photoconductor of this type leaves much room for material selection and the properties of the photoconductor can be easily controlled, and “function-separated photoconductors”, in which the functions of charge generation and transfer are shared by different compounds, are thus becoming mainstream. For example, there are known a single-layered electrophotographic photoconductor (hereinafter referred to as “single-layered photoconductor”) having a charge generation material (CGM) and a charge transport material (CTM) in the same layer, and a multi-layered electrophotographic photoconductor (hereinafter referred to as “multi-layered photoconductor”) having a charge generation layer containing a charge generation material (CGM) and a charge transport layer containing a charge transport material (CTM) laminated on each other. The photoconductor can be charged by a negative charging method, in which the surface of the photoconductor is negatively charged, or by a positive charging method, in which the surface of the photoconductor is positively charged.

The combinations of layer configurations and charging methods of the photoconductor currently in practical use include a “negatively-charged multi-layered photoconductor” and a “positively-charged single-layered photoconductor”.

The “negatively-charged multi-layered photoconductor” generally has a configuration in which an undercoat layer (UCL) made of a resin or the like is provided on a conductive substrate such as an aluminum tube, a charge generation layer (CGL) made of a charge generation material (CGM) and a resin or the like is provided thereon, and a charge transport layer (CTL) made of a hole transport material (HTM) and a resin or the like is further provided thereon.

The “positively-charged single-layered photoconductor” generally has a configuration in which an undercoat layer (UCL) made of a resin or the like is provided on a conductive substrate such as an aluminum tube, and a single-layered photosensitive layer made of a charge generation material (CGM), a hole transport material (HTM), an electron transport material (ETM), and a resin or the like is provided thereon (see, for example, Patent Literature 1).

In both types of photoconductors, the surface of the photoconductor is charged by a corona discharging method or a contact method, and the photoconductor is then exposed to light to neutralize the charge on the surface, thereby forming an electrostatic latent image by the potential difference with the surrounding surface. Thereafter, a toner is brought into contact with the surface of the photoconductor to form a toner image corresponding to the electrostatic latent image, and the toner image is transferred to paper or the like and heat-melted and fixed to complete printing.

The electrophotographic photoconductor includes, as a basic configuration, a conductive support and a photosensitive layer formed on the conductive support as described above, and further includes a protective layer provided on the photosensitive layer for the purpose of improving abrasion resistance and the like.

As technologies for improving the mechanical strength or abrasion resistance of the surface of the photoconductor, there are disclosed photoconductors in which a layer containing a chain polymerizable functional group-containing compound as a binder resin is formed on an outermost layer of the photoconductor, and is polymerized by applying energy such as heat, light, or radiation to form a cured resin layer (see, for example, Patent Literatures 1 and 2).

### CITATION LIST

### Patent Literature

Patent Literature 1: U.S. Pat. No. 9,417,538
Patent Literature 2: International Publication No. 2010/035683 (U.S. Pat. No. 5,263,296)

### SUMMARY OF THE INVENTION

### Problem to be Solved by the Invention

As described above, protective layers are being implemented to improve the abrasion resistance of photoconductors. Among them, protective layers that use curable compounds are particularly excellent in mechanical strength.

Such protective layers are required to have electron transportability from the viewpoint of improving the electrical properties of photoconductors. As a means to achieve this, it is considered effective to incorporate compounds having electron transportability into protective layers that use curable compounds. The method for achieving this includes dissolving a curable composition containing a compound having electron transportability in an organic solvent to prepare a protective layer-forming coating liquid, and coating the protective layer-forming coating liquid on the surface of the photoconductor.

However, it has been found that some compounds having electron transportability have insufficient solubility in organic solvents and insufficient electrical properties, especially residual potential properties, when incorporated into protective layers.

An object of the present invention is to provide a compound having electron transportability and sufficient solubility in organic solvents, and a novel electrophotographic photoconductor having a photosensitive layer and a protective layer in sequence on a conductive support, which can provide good electrical properties, especially residual potential properties, even when the compound having electron transportability is contained in the protective layer.

### Means for Solving Problem

As a result of investigations by the present inventors, the above problems are solved by the following means.

[1] An electrophotographic photoconductor having a photosensitive layer and a protective layer in sequence on a conductive support, wherein the protective layer contains a polymer of an electron transporting compound represented by the following formula (1).

A-X-B (1)

In the formula (1), X represents an electron transporting skeleton. A is represented by the following formula (2), B is represented by the following formula (3), and A and B are different from each other.

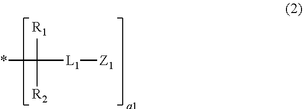

In the formula (2), the asterisk (*) represents a bond with the formula (1). R₁ and R₂ each independently represent a hydrogen atom, an optionally substituted alkyl group, an optionally substituted alkoxy group, an optionally substituted aryloxy group, an optionally substituted heteroaryloxy group, an optionally substituted alkoxycarbonyl group, an optionally substituted dialkylamino group, an optionally substituted diarylamino group, an optionally substituted arylalkylamino group, an optionally substituted acyl group, an optionally substituted haloalkyl group, an optionally substituted alkylthio group, an optionally substituted arylthio group, an optionally substituted silyl group, an optionally substituted siloxy group, an optionally substituted aromatic hydrocarbon group, or an optionally substituted aromatic heterocyclic group. L₁ represents a divalent group. Z₁ represents an optionally substituted acryloyl group, an optionally substituted methacryloyl group, an optionally substituted acrylamide group, or an optionally substituted methacrylamide group. a1 is an integer of 1 or more, and when a1 is an integer of 2 or more, each of R₁, R₂, L₁, and Z₁ may be the same or different from each other.

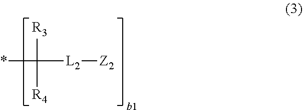

In the formula (3), the asterisk (*) represents a bond with the formula (1). R₃ and R₄ each independently represent a hydrogen atom, an optionally substituted alkyl group, an optionally substituted alkoxy group, an optionally substituted aryloxy group, an optionally substituted heteroaryloxy group, an optionally substituted alkoxycarbonyl group, an optionally substituted dialkylamino group, an optionally substituted diarylamino group, an optionally substituted arylalkylamino group, an optionally substituted acyl group, an optionally substituted haloalkyl group, an optionally substituted alkylthio group, an optionally substituted arylthio group, an optionally substituted silyl group, an optionally substituted siloxy group, an optionally substituted aromatic hydrocarbon group, or an optionally substituted aromatic heterocyclic group. L₂ represents a divalent group. Z₂ represents a hydrogen atom, an alkoxy group, or an amide group. b1 is an integer of 1 or more, and when b1 is an integer of 2 or more, each of R₃, R₄, L₂, and Z₂ may be the same or different from each other.

[2] The electrophotographic photoconductor according to the above [1], wherein X in the formula (1) is at least one selected from the group consisting of the formulae (A-1) to (A-27) described later.

In the formulae (A-1) to (A-27) described later, the asterisk (*) represents a bond with the formula (2) or (3); R_{A11}, R_{A21}, R_{A31}, R_{A41}, R_{A51}, R_{A61}, R_{A62}, R_{A71}, R_{A72}, R_{A81}, R_{A82}, R_{A91}, R_{A92}, R_{A101}, R_{A111}, R_{A121}, R_{A131}, R_{A141}, R_{A151}, R_{A161}, R_{A171}, R_{A181}, R_{A182}, R_{A191}, R_{A192}, R_{A201}, R_{A202}, R_{A211}, R_{A212}, R_{A221}, R_{A222}/R_{A231}, R_{A232}, R_{A241}, R_{A242}, R_{A251}, R_{A252}, R_{A261}, and R_{A262} each independently represent a hydrogen atom, an optionally substituted alkyl group, an optionally substituted aralkyl group, an optionally substituted aromatic group, an optionally substituted alkoxy group, an optionally substituted aryloxy group, an optionally substituted acyl group, an optionally substituted ester group, an optionally substituted cyano group, an optionally substituted nitro group, an optionally substituted sulfone group, an optionally substituted hydroxy group, an optionally substituted aldehyde group, or a halogen atom. m11, m21, m31, m41, m51, m61, m62, m71, m72, m81, m82, m91, m92, m101, m111, m121, m131, m141, m151, m161, m171, m181, m182, m191, m192, m201, m202, m211, m212, m221, m222, m231, m232, m241, m242, m251, m252, m261, and m262 each independently represent an integer of 0 or more. When m11, m21, m31, m41, m51, m61, m62, m71, m72, m81, m82, m91, m92, m101, m111, m121, m131, m141, m151, m161, m171, m181, m182, m191, m192, m201, m202, m211, m212, m221, m222, m231, m232, m241, m242, m251, m252, m261, and m262 are each 2 or more, each of R_{A11} to R_{A262} may be different from each other.

[3] The electrophotographic photoconductor according to the above [1] or [2], wherein in the formulae (2) and (3), L₁ and L₂ each independently represent an alkylene group, a divalent group having a ketone group, a divalent group having an ether bond, a divalent group having an ester bond, or a group formed by linking these groups.

[4] The electrophotographic photoconductor according to any one of the above [1] to [3], wherein in the formulae (2) and (3), when R₁ to R₄ are each other than a hydrogen atom, the substituent that R₁ to R₄ may have is an alkyl group.

[5] The electrophotographic photoconductor according to any one of the above [1] to [4], wherein the electron transporting compound represented by the formula (1) has at least two or more polymerizable functional groups.

[6] An electrophotographic photoconductor cartridge including the electrophotographic photoconductor according to any one of the above [1] to [5].

[7] An image formation device including the electrophotographic photoconductor according to any one of the above [1] to [5].

[8] A compound being represented by the following formula (1).

A-X-B (1)

In the formula (1), X represents an electron transporting skeleton. A is represented by the following formula (2), B is represented by the following formula (3), and A and B are different from each other.

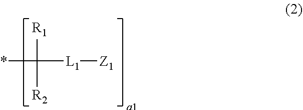

In the formula (2), the asterisk (*) represents a bond with the formula (1). R₁ and R₂ each independently represent a hydrogen atom, an optionally substituted alkyl group, an optionally substituted alkoxy group, an optionally substituted aryloxy group, an optionally substituted heteroaryloxy group, an optionally substituted alkoxycarbonyl group, an optionally substituted dialkylamino group, an optionally substituted diarylamino group, an optionally substituted arylalkylamino group, an optionally substituted acyl group, an optionally substituted haloalkyl group, an optionally substituted alkylthio group, an optionally substituted arylthio group, an optionally substituted silyl group, an optionally substituted siloxy group, an optionally substituted aromatic hydrocarbon group, or an optionally substituted aromatic heterocyclic group. L₁ represents a divalent group. Z₁ represents an optionally substituted acryloyl group, an optionally substituted methacryloyl group, an optionally substituted acrylamide group, or an optionally substituted methacrylamide group. a1 is an integer of 1 or more, and when a1 is an integer of 2 or more, each of R₁, R₂, L₁, and Z₁ may be the same or different from each other.

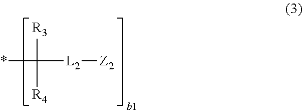

In the formula (3), the asterisk (*) represents a bond with the formula (1). R₃ and R₄ each independently represent a hydrogen atom, an optionally substituted alkyl group, an optionally substituted alkoxy group, an optionally substituted aryloxy group, an optionally substituted heteroaryloxy group, an optionally substituted alkoxycarbonyl group, an optionally substituted dialkylamino group, an optionally substituted diarylamino group, an optionally substituted arylalkylamino group, an optionally substituted acyl group, an optionally substituted haloalkyl group, an optionally substituted alkylthio group, an optionally substituted arylthio group, an optionally substituted silyl group, an optionally substituted siloxy group, an optionally substituted aromatic hydrocarbon group, or an optionally substituted aromatic heterocyclic group. L₂ represents a divalent group. Z₂ represents a hydrogen atom, an alkoxy group, or an amide group. b1 is an integer of 1 or more, and when b1 is an integer of 2 or more, each of R₃, R₄, L₂, and Z₂ may be the same or different from each other.

[9] The compound according to the above [8], wherein X in the formula (1) is at least one selected from the group consisting of the formulae (A-1) to (A-27) described later.

In the formulae (A-1) to (A-27) described later, the asterisk (*) represents a bond with the formula (2) or (3); R_{A11}, R_{A21}, R_{A31}, R_{A41}, R_{A51}, R_{A61}, R_{A62}, R_{A71}, R_{A72}, R_{A81}, R_{A82}, R_{A91}, R_{A92}, R_{A101}, R_{A111}, R_{A121}, R_{A131}, R_{A141}, R_{A151}, R_{A161}, R_{A171}, R_{A181}, R_{A182}, R_{A191}, R_{A192}, R_{A201}, R_{A202}, R_{A211}, R_{A212}, R_{A221}, R_{A222}/R_{A231}, R_{A232}, R_{A241}, R_{A242}, R_{A251}, R_{A252}, R_{A261}, and R_{A262} each independently represent a hydrogen atom, an optionally substituted alkyl group, an optionally substituted aralkyl group, an optionally substituted aromatic group, an optionally substituted alkoxy group, an optionally substituted aryloxy group, an optionally substituted acyl group, an optionally substituted ester group, an optionally substituted cyano group, an optionally substituted nitro group, an optionally substituted sulfone group, an optionally substituted hydroxy group, an optionally substituted aldehyde group, or a halogen atom. m11, m21, m31, m41, m51, m61, m62, m71, m72, m81, m82, m91, m92, m101, m111, m121, m131, m141, m151, m161, m171, m181, m182, m191, m192, m201, m202, m211, m212, m221, m222, m231, m232, m241, m242, m251, m252, m261, and m262 each independently represent an integer of 0 or more. When m11, m21, m31, m41, m51, m61, m62, m71, m72, m81, m82, m91, m92, m101, m111, m121, m131, m141, m151, m161, m171, m181, m182, m191, m192, m201, m202, m211, m212, m221, m222, m231, m232, m241, m242, m251, m252, m261, and m262 are each 2 or more, each of R_{A11} to R_{A262} may be different from each other.

The compound according to the above [8] or [9], wherein in the formulae (2) and (3), L₁ and L₂ each independently represent an alkylene group, a divalent group having a ketone group, a divalent group having an ether bond, a divalent group having an ester bond, or a group formed by linking these groups.

The compound according to any one of the above [8] to [10], wherein in the formulae (2) and (3), when R₁ to R₄ are each other than a hydrogen atom, the substituent that R₁ to R₄ may have is an alkyl group.

The compound according to any one of the above [8] to [11], having at least two or more polymerizable functional groups.

### Effect of the Invention

The compound proposed by the present invention has a left-right asymmetric structure in which an electron transporting skeleton is centered, with a side chain having a polymerizable functional group on one side, and with a side chain having no polymerizable functional group on the other side, and thus has electron transportability and sufficient solubility in organic solvents. Therefore, the compound is useful, for example, as an electron transporting compound used as a raw material for an electrophotographic photoconductor used in printers, copiers, and the like.

The electrophotographic photoconductor proposed by the present invention contains, in the protective layer, a polymer of an electron transporting compound having a left-right asymmetric structure in which an electron transporting skeleton is centered, with a side chain having a polymerizable functional group on one side, and with a side chain having no polymerizable functional group on the other side, thereby improving the electron transportability in the protective layer and the electrical properties, especially the residual potential properties.

### BRIEF DESCRIPTION OF DRAWINGS

FIG. 1 is a schematic diagram of an example configuration of an image formation device that can be configured using an electrophotographic photoconductor according to one example of the present invention.

### MODE(S) FOR CARRYING OUT THE INVENTION

Modes for carrying out the present invention (hereinafter referred to as embodiments of the present invention) are described in detail below. The present invention is not to be limited to the following embodiments, and may be implemented with various modifications within the scope of the gist thereof.

### <<Present Electrophotographic Photoconductor>>

The electrophotographic photoconductor according to an example of the embodiments of the present invention (also referred to as “the present electrophotographic photoconductor”) is an electrophotographic photoconductor including a photosensitive layer and a protective layer in sequence on a conductive support.

The present electrophotographic photoconductor may optionally include other layers in addition to the photosensitive layer and the protective layer.

The charging method of the present electrophotographic photoconductor may be either a negative charging method in which the surface of the photoconductor is negatively charged, or a positive charging method in which the surface of the photoconductor is positively charged. Among them, the positive charging method is preferred because it is considered that the effects of the present invention can be obtained more effectively with the positive charging method from the viewpoint of seeking the electron transportability in the protective layer.

In the present electrophotographic photoconductor, the side opposite to the conductive support is an upper side or a front surface side, and the conductive support side is a lower side or a back surface side.

### <Present Protective Layer>

The present protective layer is preferably a layer containing an electron transporting compound. When the electron transporting compound has a polymerizable functional group, such as a compound represented by the formula (1) described later, the present protective layer is preferably a layer containing a polymer of the electron transporting compound. That is, the present protective layer is preferably a layer containing a cured product obtained by curing the electron transporting compound.

The present protective layer is preferably a layer further containing a cured product obtained by curing a curable compound. That is, the present protective layer is preferably a layer containing polymers of an electron transporting compound and a curable compound, in other words, a layer containing cured products obtained by curing an electron transporting compound and a curable compound.

The “electron transporting compound” means a compound having electron transportability, in other words, a compound having an electron transporting skeleton.

The present protective layer can be formed, for example, from a composition containing an electron transporting compound and optionally a curable compound, a polymerization initiator, inorganic particles, and other materials. However, the present protective layer is not limited to those formed from such a composition.

From the viewpoint of further obtaining the effect of the present invention, the present protective layer is preferably an outermost layer, i.e., an outermost layer located on the side opposite to the conductive support. However, the effect of the present invention can be obtained even when the protective layer is not necessarily the outermost layer. For example, if there is some kind of segregation layer is present on the outermost layer of the photoconductor, the effect can also be obtained even when the protective layer is not the outermost layer.

### (Electron Transporting Compound)

The electron transporting compound used in the present protective layer is preferably a compound proposed by the present invention. Specifically, it is preferably a compound represented by the following formula (1). Such a compound is an electron transporting compound having a left-right asymmetric structure in which an electron transporting skeleton is centered, with a side chain having a polymerizable functional group on one side, and with a side chain having no polymerizable functional group on the other side. By containing such an electron transporting compound in the protective layer, the electron transportability in the protective layer is improved, resulting in good electrical properties of the photoconductor. In addition, because the molecule has low symmetry, the electron transporting compound has low crystallinity, resulting in good solubility in organic solvents.

In addition, the highly polar structure of the formulae (2) and/or (3) described below provides better solubility in organic solvents, especially polar organic solvents.

A-X-B (1)

### [Formula (1)]

In the formula (1), X represents an electron transporting skeleton. The above X is described in more detail later.

In the formula (1), A is preferably a structure represented by the following formula (2), and B is preferably a structure represented by the following formula (3).

The electron transporting compound represented by the formula (1) preferably has at least two or more polymerizable functional groups from the viewpoint of curability, and from the viewpoint of stability, it preferably has 8 or less, more preferably 6 or less, and even more preferably 4 or less polymerizable functional groups.

### [Formula (2)]

The formula (2) is an example of a preferred structure of A in the formula (1), as described above.

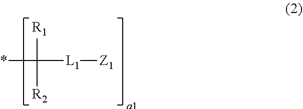

The formula (2) represents a side chain having a polymerizable functional group, and the asterisk (*) represents a bond with the formula (1).

It is preferred that R₁ and R₂ are each independently a hydrogen atom, an optionally substituted alkyl group, an optionally substituted alkoxy group, an optionally substituted aryloxy group, an optionally substituted heteroaryloxy group, an optionally substituted alkoxycarbonyl group, an optionally substituted dialkylamino group, an optionally substituted diarylamino group, an optionally substituted arylalkylamino group, an optionally substituted acyl group, an optionally substituted haloalkyl group, an optionally substituted alkylthio group, an optionally substituted arylthio group, an optionally substituted silyl group, an optionally substituted siloxy group, an optionally substituted aromatic hydrocarbon group, or an optionally substituted aromatic heterocyclic group.

When R₁ and R₂ are each independently other than a hydrogen atom, the substituent that R₁ and R₂ may have is preferably an alkyl group from the viewpoint of solubility.

In the present invention, the term “optionally substituted” means that the group may have a substituent and includes both the case of having a substituent and the case of not having a substituent.

In the compound of the present invention, examples of the substituent such as the optionally substituted alkyl group include an alkyl group, an alkoxy group, an aryloxy group, a heteroaryloxy group, an alkoxycarbonyl group, a dialkylamino group, a diarylamino group, an arylalkylamino group, an acyl group, a haloalkyl group, an alkylthio group, an arylthio group, a silyl group, a siloxy group, an aromatic hydrocarbon group, and an aromatic heterocyclic group. However, when these groups have a substituent, the substituent is preferably an alkyl group, and it is more preferred that these groups have no substituent, from the viewpoint of solubility.

In the formula (2), L₁ may be a divalent group. Examples thereof include an alkylene group, a divalent group having a ketone group, a divalent group having an ether bond, a divalent group having an ester bond, and a group formed by linking these groups. However, it is not limited to these.

The divalent group having an ester bond is preferably a group represented by the following formula (E-1) or (E-2).

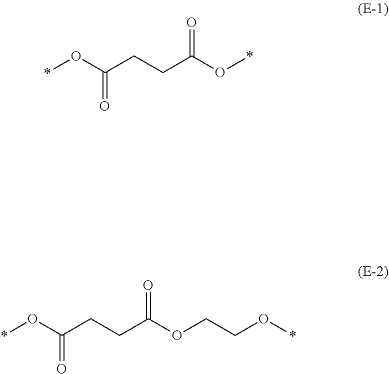 [SMILES: *OC(=O)CCC(=O)O* ; *OCCOC(=O)CCC(=O)O*]

In the formulae (E-1) and (E-2), the asterisk (*) represents a bonding site with the carbon to which R₁ and R₂ are bonded and a bonding site with Z₁.

Among them, the formula (E-1) is preferred from the viewpoint of solubility and stability.

In the formula (2), Z₁ is preferably an optionally substituted acryloyl group, an optionally substituted methacryloyl group, an optionally substituted acrylamide group, or an optionally substituted methacrylamide group. The optionally substituted acryloyl group and the optionally substituted methacryloyl group may be groups represented by the following formulae (P-1) to (P-5).

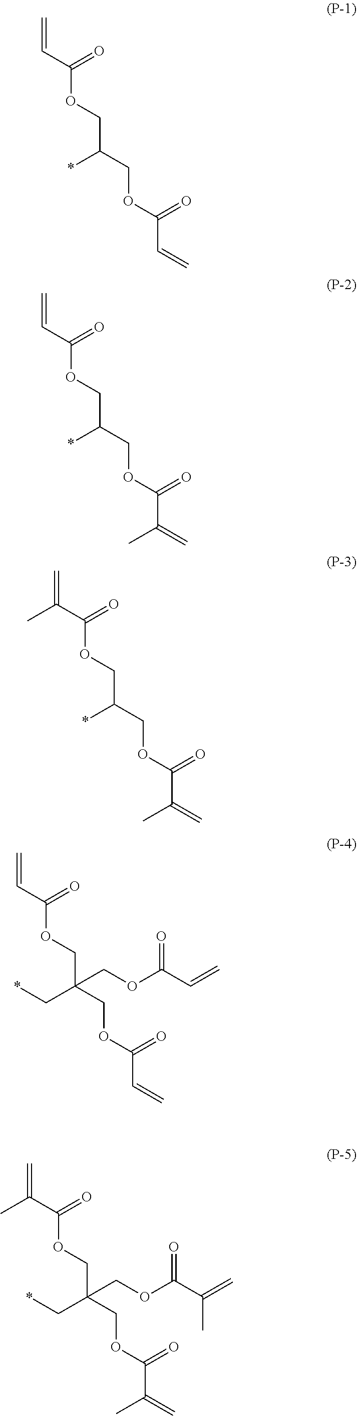

In the formulae (P-1) to (P-5), the asterisk (*) represents a bonding site with L₁.

Among them, the formula (P-3) is preferred from the viewpoint of solubility and stability.

In the formula (2), a1 is an integer of 1 or more, and it is preferably 4 or less, more preferably 3 or less, and even more preferably 2 or less.

When a1 is an integer of 2 or more, each of R₁, R₂, L₁, and Z₁ may be the same or different from each other.

### [Formula (3)]

The formula (3) is an example of a preferred structure of B in the formula (1), as described above.

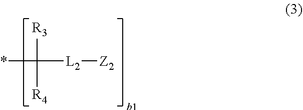

The formula (3) represents a side chain having no polymerizable functional group, and the asterisk (*) represents a bond with the formula (1).

It is preferred that R₃ and R₄ are each independently a hydrogen atom, an optionally substituted alkyl group, an optionally substituted alkoxy group, an optionally substituted aryloxy group, an optionally substituted heteroaryloxy group, an optionally substituted alkoxycarbonyl group, an optionally substituted dialkylamino group, an optionally substituted diarylamino group, an optionally substituted arylalkylamino group, an optionally substituted acyl group, an optionally substituted haloalkyl group, an optionally substituted alkylthio group, an optionally substituted arylthio group, an optionally substituted silyl group, an optionally substituted siloxy group, an optionally substituted aromatic hydrocarbon group, or an optionally substituted aromatic heterocyclic group.

When R₃ and R₄ are each independently other than a hydrogen atom, the substituent that R₃ and R₄ may have is preferably an alkyl group from the viewpoint of solubility.

L₂ may be a divalent group. Examples thereof include an alkylene group, a divalent group having a ketone group, a divalent group having an ether bond, a divalent group having an ester bond, and a group formed by linking these groups. However, it is not limited to these.

The divalent group having an ester bond is preferably a group represented by the following formula (E-1) or (E-2).

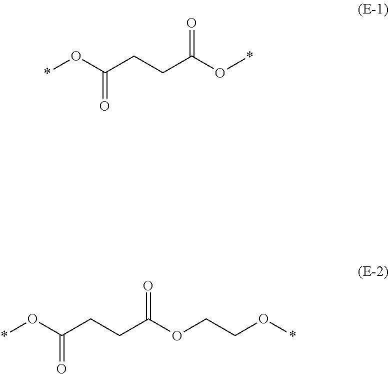 [SMILES: *OC(=O)CCC(=O)O* ; *OCCOC(=O)CCC(=O)O*]

In the formulae (E-1) and (E-2), the asterisk (*) represents a bonding site with the carbon to which R₃ and R₄ are bonded and a bonding site with Z₂.

Z₂ is preferably a hydrogen atom, an optionally substituted alkoxy group, or an optionally substituted amide group.

b1 is an integer of 1 or more, and it is preferably 4 or less, more preferably 3 or less, and even more preferably 2 or less.

When b1 is an integer of 2 or more, each of R₃, R₄, L₂, and Z₂ may be the same or different from each other.

The side chain having no polymerizable functional group represented by the formula (3) preferably has a branched structure. It is presumed that the branching causes more significant steric hindrance and reduces crystallinity, resulting in even better solubility in solvents, especially alcohols.

### [X]

Examples of X in the formula (1), i.e., the electron transporting skeleton include at least one skeleton selected from the group consisting of the following formulae (A-1) to (A-27).

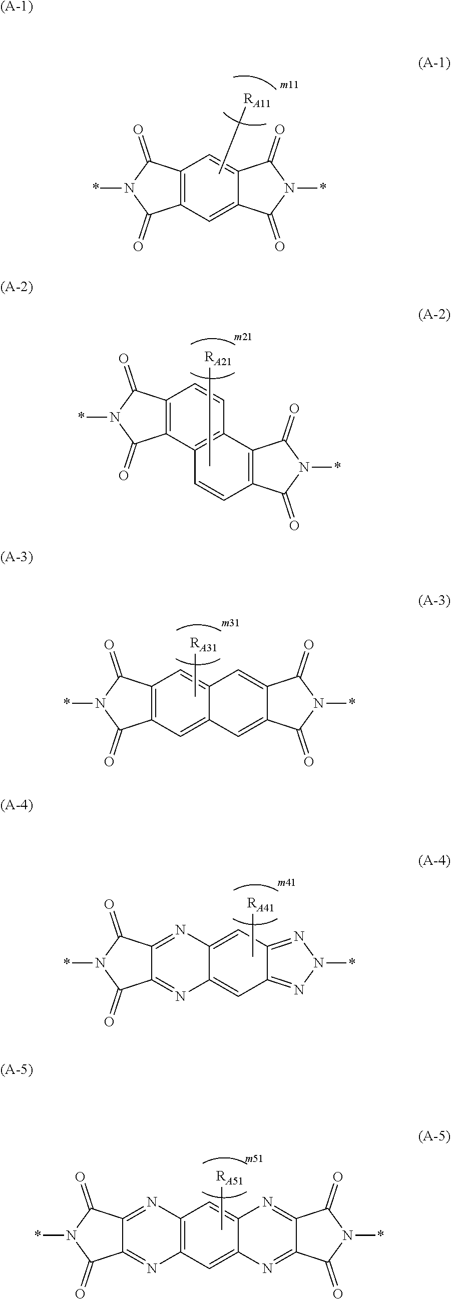

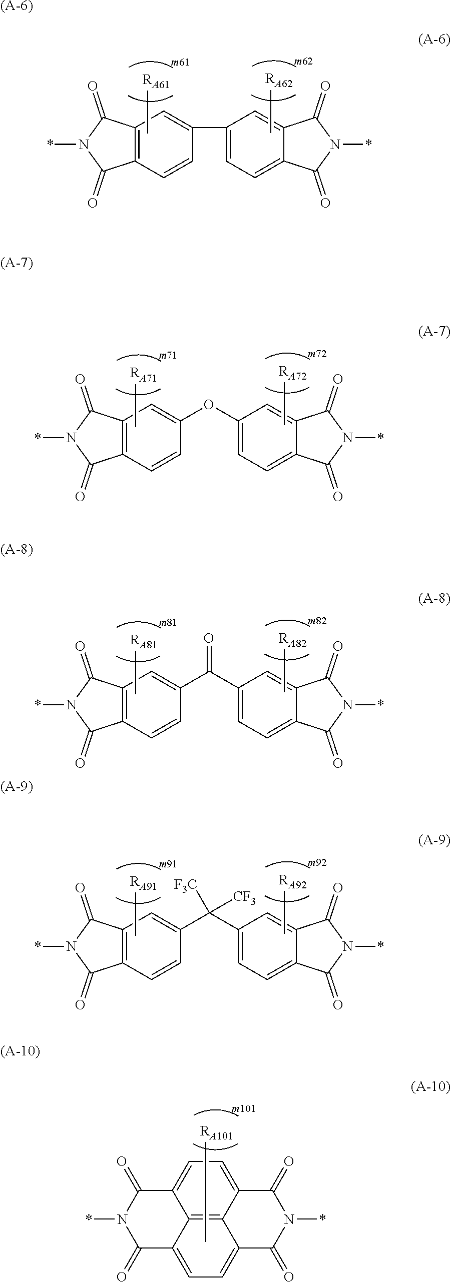

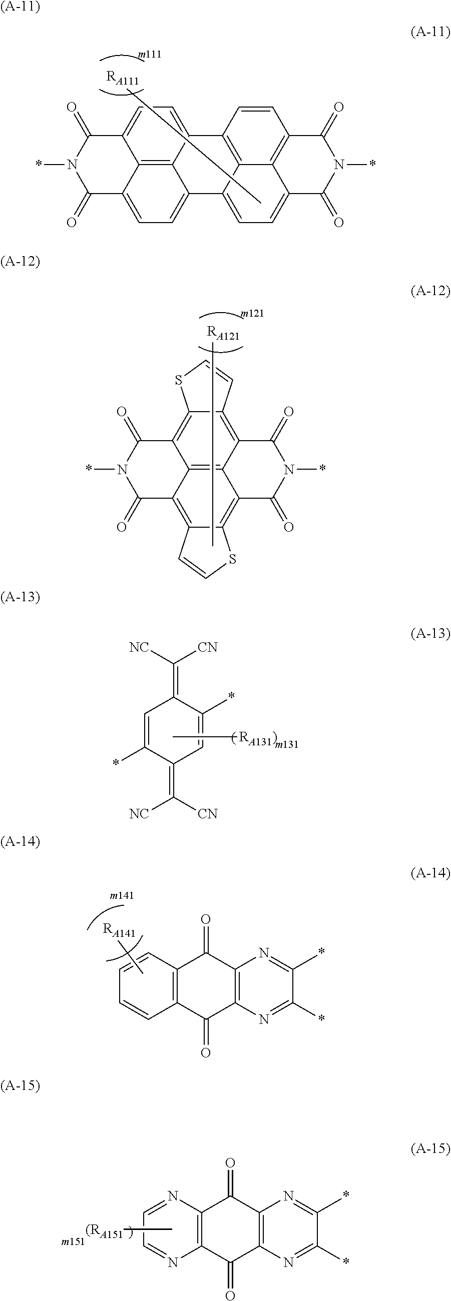

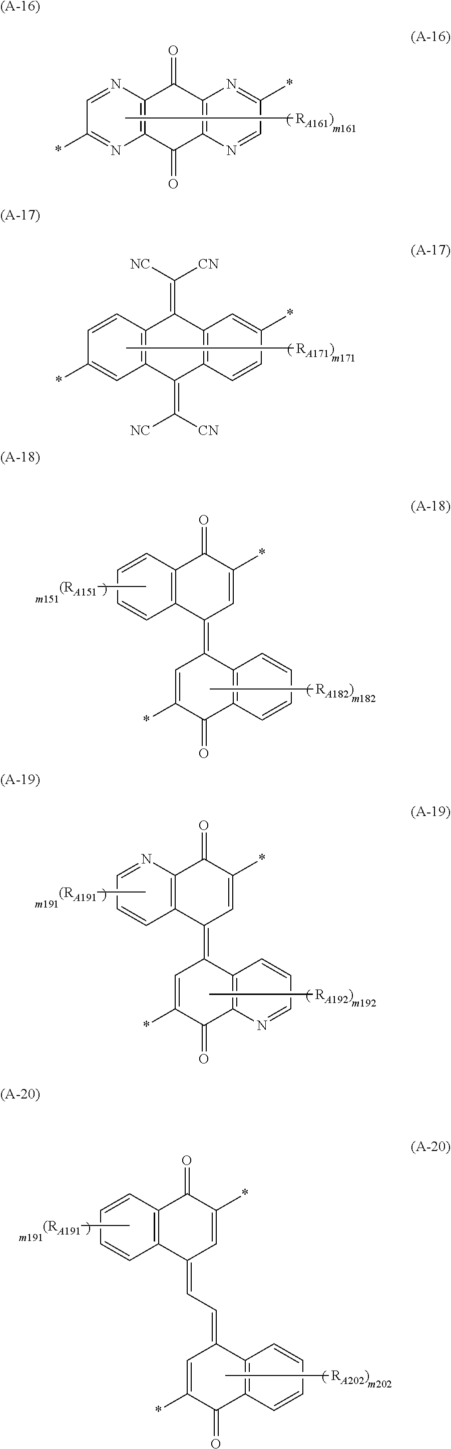

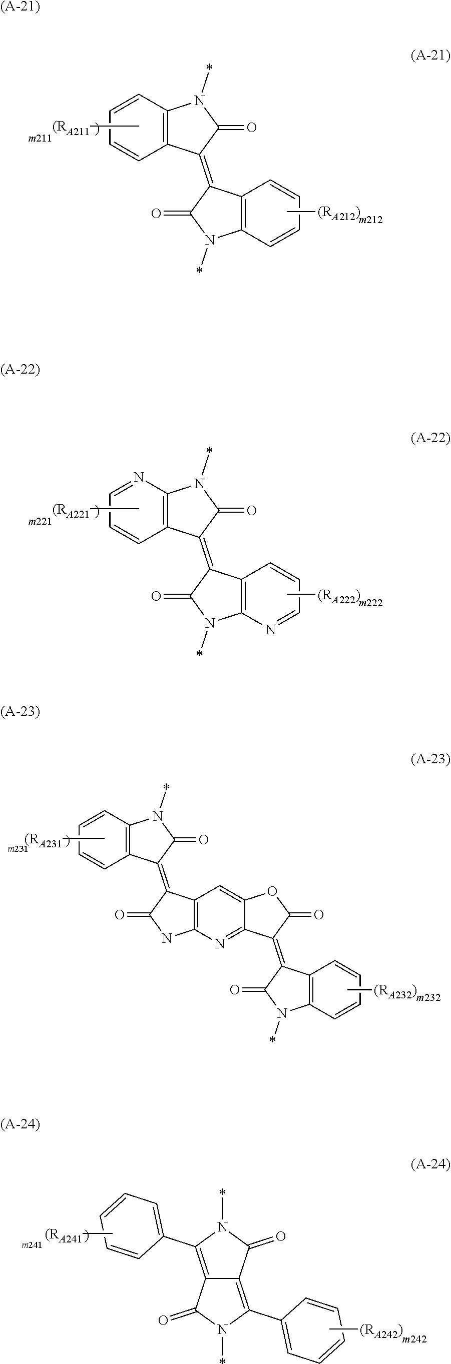

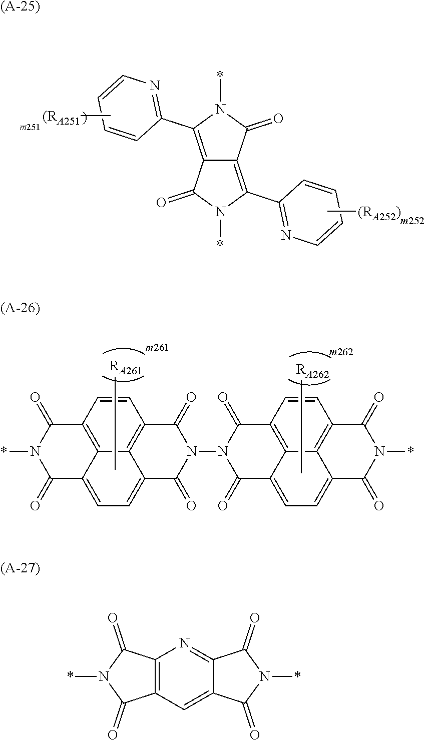

In the formulae (A-1) to (A-27), the asterisk (*) represents a bond with the formula (2) or (3).

In the formulae (A-1) to (A-27), it is preferred that R_{A11}, R_{A21}, R_{A31}, R_{A41}, R_{A51}, R_{A61}, R_{A62}, R_{A71}, R_{A72}, R_{A81}, R_{A82}, R_{A91}, R_{A92}, R_{A101}, R_{A111}, R_{A121}, R_{A131}, R_{A141}, R_{A151}, R_{A161}, R_{A171}, R_{A181}, R_{A182}, R_{A191}, R_{A192}, R_{A201}, R_{A202}, R_{A211}, R_{A212}, R_{A221}, R_{A222}, R_{A231}, R_{A232}, R_{A241}, R_{A242}, R_{A251}, R_{A252}, R_{A261}, and R_{A262} are each independently a hydrogen atom, an optionally substituted alkyl group, an optionally substituted aralkyl group, an optionally substituted aromatic group, an optionally substituted alkoxy group, an optionally substituted aryloxy group, an optionally substituted acyl group, an optionally substituted ester group, an optionally substituted cyano group, an optionally substituted nitro group, an optionally substituted sulfone group, an optionally substituted hydroxy group, an optionally substituted aldehyde group, or a halogen atom.

In the formulae (A-1) to (A-27), m11, m21, m31, m41, m51, m61, m62, m71, m72, m81, m82, m91, m92, m101, m111, m121, m131, m141, m151, m161, m171, m181, m182, m191, m192, m201, m202, m211, m212, m221, m222, m231, m232, m241, m242, m251, m252, m261, and m262 may each independently be an integer of 0 or more, and from the viewpoint of stability, each of them is preferably an integer of 3 or less, more preferably 2 or less, and even more preferably 1 or less.

When m11, m21, m31, m41, m51, m61, m62, m71, m72, m81, m82, m91, m92, m101, m111, m121, m131, m141, m151, m161, m171, m181, m182, m191, m192, m201, m202, m211, m212, m221, m222, m231, m232, m241, m242, m251, m252, m261, and m262 are each 2 or more, each of R_{A11} to R_{A262} may be different from each other.

Among them, from the viewpoint of electron transportability and stability, the formulae (A-1), (A-10), and (A-11) are preferred, and the formulae (A-10) and (A-11) are more preferred.

The content of the electron transporting compound in the present protective layer is preferably 40 parts by mass or more, more preferably 60 parts by mass or more, and even more preferably 80 parts by mass or more, relative to a total mass of the present protective layer being 100 parts by mass, from the viewpoint of electron transportability.

The electron transporting compound in the present protective layer may also contain other electron transporting compounds different from the compound represented by the formula (1).

The content of the compound represented by the formula (1) in the present protective layer is preferably 40 parts by mass or more, more preferably 50 parts by mass or more, and even more preferably 60 parts by mass or more, relative to a total mass of the electron transporting compound in the present protective layer being 100 parts by mass, from the viewpoint of solubility.

The following describes specific examples of the present electron transporting compound. However, it is not limited to these.

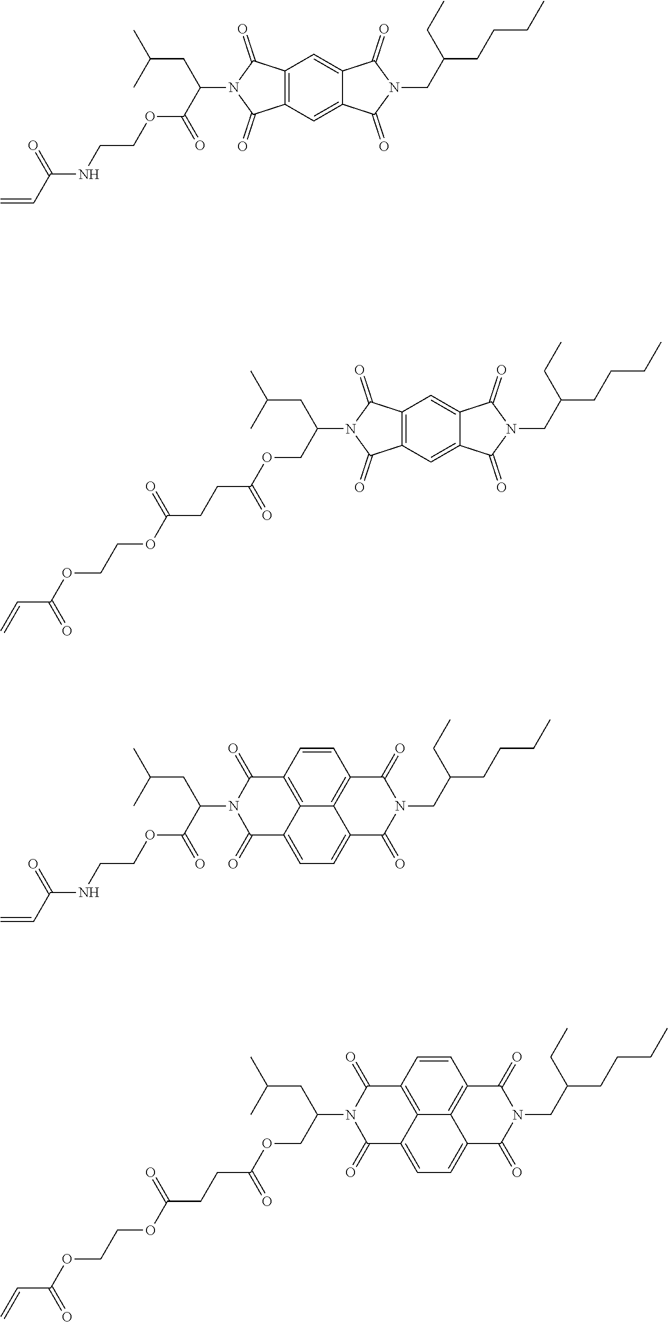

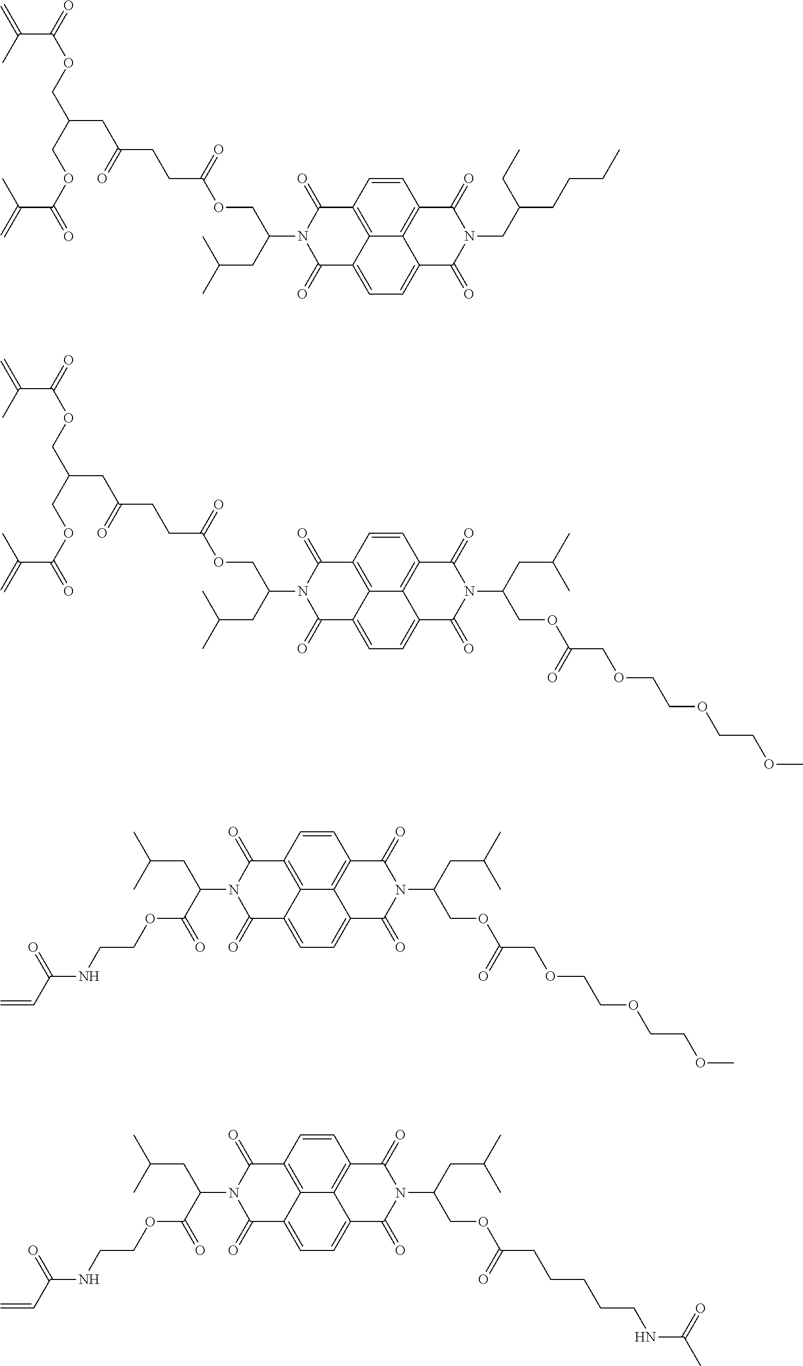

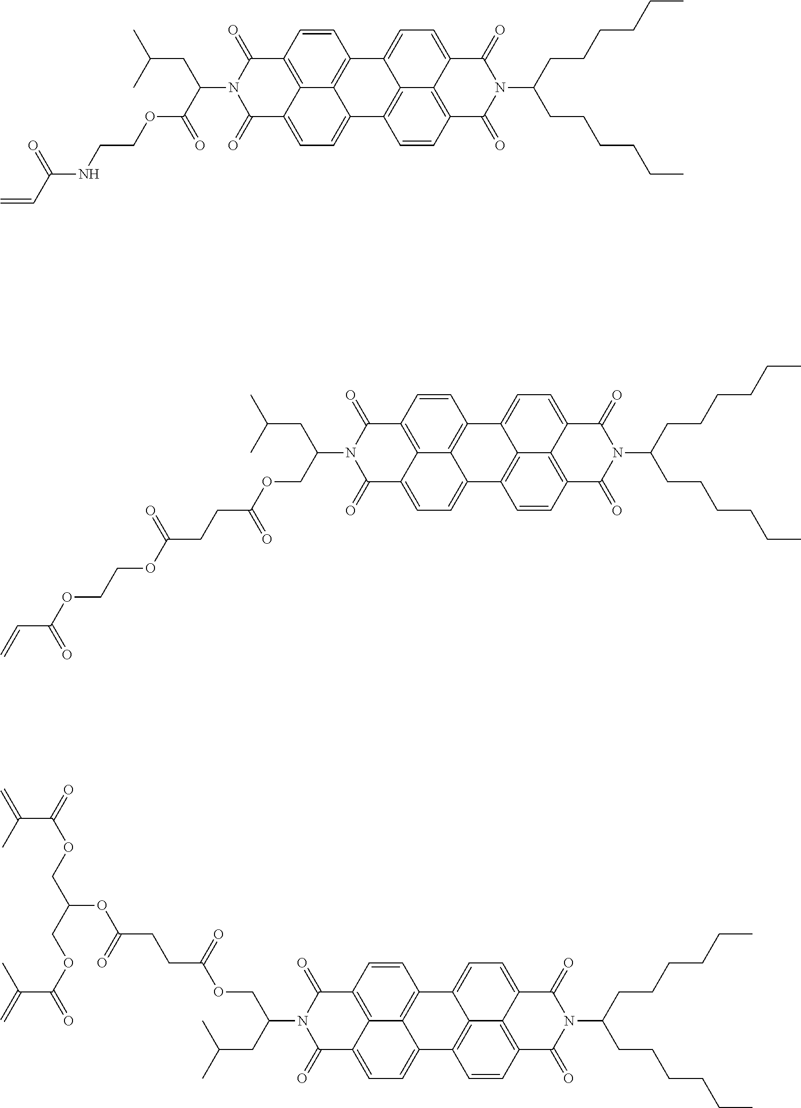

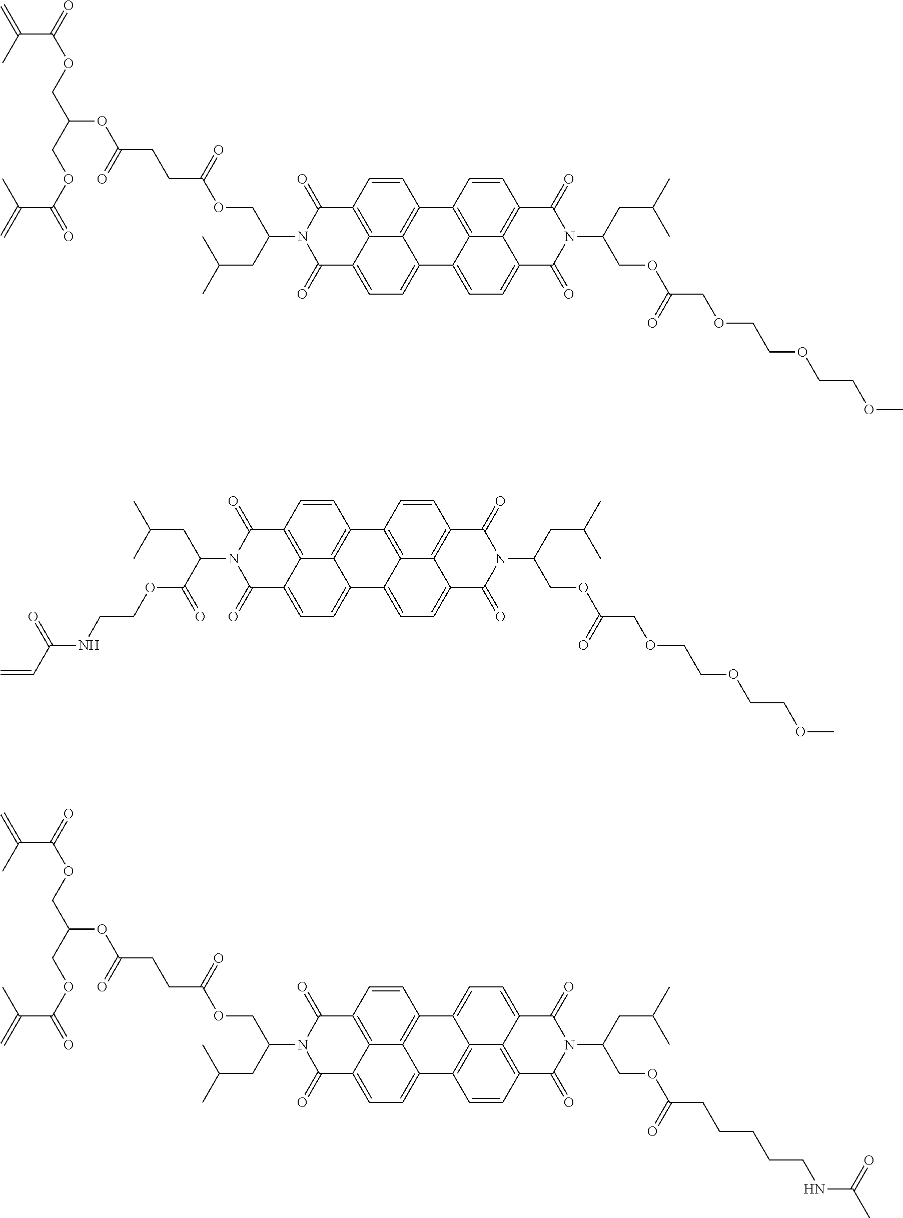

### (Curable Compound)

The curable compound may be any compound having a chain polymerizable functional group. Among them, monomers, oligomers, or polymers having a radical polymerizable functional group are preferred. Among them, curable compounds having crosslinking properties, especially photocurable compounds, are preferred. Examples thereof include curable compounds having two or more radical polymerizable functional groups. Compounds having one radical polymerizable functional group may be used in combination.

The radical polymerizable functional groups may include either acryloyl groups (including acryloyloxy groups) or methacryloyl groups (including methacryloyloxy groups), or both of these groups.

The following are examples of preferred compounds as curable compounds having radical polymerizable functional groups.

Examples of the monomer having an acryloyl group or a methacryloyl group include trimethylolpropane triacrylate (TMPTA), trimethylolpropane trimethacrylate, pentaerythritol triacrylate, pentaerythritol tetraacrylate, glycerol triacrylate, tris(acryloxyethyl) isocyanurate, dipentaerythritol hexaacrylate, dimethylolpropane tetraacrylate, pentaerythritol ethoxytetraacrylate, EO-modified phosphate triacrylate, 2,2,5,5-tetrahydroxymethylcyclopentanone tetraacrylate, 2-hydroxy-3-acryloyloxypropyl methacrylate, polyethylene glycol diacrylate, polypropylene glycol diacrylate, polytetramethylene glycol diacrylate, EO-modified bisphenol A diacrylate, PO-modified bisphenol A diacrylate, 9,9-bis[4-(2-acryloyloxyethoxy)phenyl]fluorene, tricyclodecane dimethanol diacrylate, decanediol diacrylate, hexanediol diacrylate, ethylene glycol dimethacrylate, polyethylene glycol dimethacrylate, EO-modified bisphenol A dimethacrylate, PO-modified bisphenol A dimethacrylate, tricyclodecane dimethanol dimethacrylate, decanediol dimethacrylate, and hexanediol dimethacrylate.

Examples of the oligomer and the polymer having an acryloyl group or a methacryloyl group include urethane acrylate, ester acrylate, acrylic acrylate, and epoxy acrylate. Among them, urethane acrylate and ester acrylate are preferred, and ester acrylate is more preferred.

These curable compounds may be used alone or in a combination of two or more types.

The content ratio (mass ratio) of the curable compound to the electron transporting compound in the present protective layer is preferably 1.0 or less, more preferably 0.5 or less, and even more preferably 0.1 or less, from the viewpoint of electron transportability.

### (Polymerization Initiator)

Examples of the polymerization initiator include a thermal polymerization initiator and a photopolymerization initiator.

Examples of the thermal polymerization initiator include peroxide-based compounds such as 2,5-dimethylhexane-2,5-dihydroperoxide, and azo-based compounds such as 2,2′-azobis(isobutyronitrile).

The photopolymerization initiator can be classified into a direct cleavage type and a hydrogen abstraction type based on the difference in radical generation mechanism.

The direct cleavage type photopolymerization initiator generates radicals by cleaving some of the covalent bonds in the molecule upon absorption of light energy. The hydrogen abstraction type photopolymerization initiator generates radicals by abstracting hydrogen from the hydrogen donor by excited molecules upon absorption of light energy.

Examples of the direct cleavage type photopolymerization initiator include: acetophenone-based or ketal-based compounds such as acetophenone, 2-benzoyl-2-propanol, 1-benzoylcyclohexanol, 2,2-diethoxyacetophenone, benzyl dimethyl ketal, and 2-methyl-4′-(methylthio)-2-morpholinopropiophenone; benzoin ether-based compounds such as benzoin, benzoin methyl ether, benzoin ethyl ether, benzoin isobutyl ether, benzoin isopropyl ether, and 0-tosylbenzoin; and acylphosphine oxide-based compounds such as diphenyl(2,4,6-trimethylbenzoyl) phosphine oxide, phenylbis(2,4,6-trimethylbenzoyl) phosphine oxide, and lithium phenyl(2,4,6-trimethylbenzoyl)phosphonate.

Examples of the hydrogen abstraction type photopolymerization initiator include: benzophenone-based compounds such as benzophenone, 4-benzoylbenzoic acid, 2-benzoylbenzoic acid, methyl 2-benzoylbenzoate, methyl benzoylformate, benzyl, p-anisyl, 2-benzoylnaphthalene, 4,4′-bis(dimethylamino)benzophenone, 4,4′-dichlorobenzophenone, and 1,4-dibenzoylbenzene; and anthraquinone-based or thioxanthone-based compounds such as 2-ethylanthraquinone, 2-isopropylthioxanthone, 2-chlorothioxanthone, 2,4-dimethylthioxanthone, 2,4-diethylthioxanthone, and 2,4-dichlorothioxanthone.

Examples of other photopolymerization initiators include camphorquinone, 1-phenyl-1,2-propanedione-2-(0-ethoxycarbonyl) oxime, acridine-based compounds, triazine-based compounds, and imidazole-based compounds.

The photopolymerization initiator preferably has an absorption wavelength within the wavelength range of the light source used for light irradiation in order to efficiently absorb light energy to generate radicals. Among them, it is preferred to contain an acylphosphine oxide-based compound having an absorption wavelength on a relatively long wavelength side.

It is more preferred to use an acylphosphine oxide-based compound and a hydrogen abstraction type initiator in combination, from the viewpoint of complementing the curability of the surface of the protective layer. In this case, the content ratio of the hydrogen abstraction type initiator to the acylphosphine oxide-based compound is not particularly limited. It is preferably 0.1 part by mass or more relative to 1 part by mass of the acylphosphine oxide-based compound from the viewpoint of complementing the surface curability, and it is preferably 5 parts by mass or less from the viewpoint of maintaining the internal curability.

Materials having a photopolymerization promoting effect can be used alone or in combination with the above-mentioned photopolymerization initiator. Examples of the materials having a photopolymerization promoting effect include triethanolamine, methyldiethanolamine, ethyl 4-dimethylaminobenzoate, isoamyl 4-dimethylaminobenzoate, (2-dimethylamino)ethyl benzoate, and 4,4′-dimethylaminobenzophenone.

The polymerization initiator may be used alone or in a mixture of two or more types.

The content of the polymerization initiator is preferably 0.5 to 40 parts by mass, more preferably 1 part by mass or more or 20 parts by mass or less, relative to 100 parts by mass of the total content having radical polymerizability.

The total content having radical polymerizability includes the electron transporting compound represented by the formula (1) and the curable compound.

### (Inorganic Particles)

The present protective layer may contain inorganic particles from the viewpoint of improving the strong exposure properties and mechanical strength, or from the viewpoint of imparting charge transport capability. However, it may not contain inorganic particles.

One of the features of the present invention is that by containing a specific electron transporting compound in the present protective layer, it is not necessary to contain inorganic particles.

Examples of the inorganic particles include metal powder, metal oxide, metal fluoride, potassium titanate, and boron nitride, and in general, any inorganic particles that can be used for electrophotographic photoconductors can be used.

The inorganic particles may be used alone or in a mixture of a plurality of types.

### (Other Materials)

The present protective layer may contain other materials as necessary. Examples of the other materials include stabilizers (such as a heat stabilizer, ultraviolet absorber, light stabilizer, and antioxidant), dispersants, antistatic agents, colorants, and lubricants. These materials may be appropriately used alone or in any combination of two or more types in any ratio.

### (Method for Forming Present Protective Layer)

### [Protective Layer-Forming Coating Liquid]

The present protective layer can be formed, for example, by coating a coating liquid (referred to as “the present protective layer-forming coating liquid”), in which a curable composition containing an electron transporting compound and optionally a curable compound, a polymerization initiator, inorganic particles, and other materials is dissolved in a solvent or dispersed in a dispersion medium, onto the present photosensitive layer, and then curing the coating liquid. However, it is not limited to such a method.

The electron transporting compound, when represented by the formula (1), has a chain polymerizable functional group such as an acrylamide group, a methacrylamide group, an acryloyl group, or a methacryloyl group, and thus can also function as a curable compound. In this case, it is not necessary to contain a curable compound in addition to the electron transporting compound. Even when no curable compound is contained or the content of the curable compound is small, the use of the electron transporting compound provides sufficient mechanical strength of the protective layer and suppresses deterioration of the residual potential due to the presence of the curable compound. However, this does not preclude the use of a combination of the electron transporting compound and the curable compound.

The electron transporting compound used in the present protective layer-forming coating liquid is preferably a compound represented by the formula (1).

The preferred embodiments of the curable compound, polymerization initiator, inorganic particles, and other materials used in the present protective layer-forming coating liquid are the same as those of the materials used in the present protective layer.

The content ratio of the curable compound to the electron transporting compound (curable compound/electron transporting compound) in the present protective layer-forming coating liquid is the same as the content ratio of the curable compound to the electron transporting compound (curable compound/electron transporting compound) in the protective layer described above.

The content of the electron transporting compound in the present protective layer-forming coating liquid is preferably 4 parts by mass or more, more preferably 6 parts by mass or more, and even more preferably 8 parts by mass or more, relative to 100 parts by mass of the solvent, from the viewpoint of film uniformity of the protective layer. From the viewpoint of solubility, it is preferably 14 parts by mass or less, more preferably 12 parts by mass or less, and even more preferably 10 parts by mass or less, relative to 100 parts by mass of the solvent.

The content of the curable compound in the present protective layer-forming coating liquid is preferably 10 parts by mass or less, more preferably 5 parts by mass or less, and even more preferably 0 parts by mass, relative to 100 parts by mass of the solvent, from the viewpoint of residual potential.

As the solvent used in the present protective layer-forming coating liquid, for example, an organic solvent can be used. Examples of the organic solvent include: alcohols such as methanol, ethanol, propanol, and 2-methoxyethanol; ethers such as tetrahydrofuran, 1,4-dioxane, and dimethoxyethane; esters such as methyl formate and ethyl acetate; ketones such as acetone, methyl ethyl ketone, and cyclohexanone; aromatic hydrocarbons such as benzene, toluene, xylene, and anisole; chlorinated hydrocarbons such as dichloromethane, chloroform, 1,2-dichloroethane, 1,1,2-trichloroethane, 1,1,1-trichloroethane, tetrachloroethane, 1,2-dichloropropane, and trichloroethylene; nitrogen-containing compounds such as n-butylamine, isopropanolamine, diethylamine, triethanolamine, ethylenediamine, and triethylenediamine; and aprotic polar solvents such as acetonitrile, N-methylpyrrolidone, N, N-dimethylformamide, and dimethylsulfoxide. Mixed solvents of these can also be used in any combination and ratio. Among them, from the viewpoint of solubility and coatability, alcohols, ethers, aromatic hydrocarbons, and aprotic polar solvents are preferred; alcohols, ethers, and aromatic hydrocarbons are more preferred; alcohols and ethers are even more preferred; and alcohols are most preferred.

Even an organic solvent that alone does not dissolve the electron transporting compound used in the protective layer of the present electrophotographic photoconductor can be used if it can be dissolved, for example, by mixing with the above-mentioned organic solvents. In general, the use of mixed solvents reduces coating unevenness. When an immersion coating method is used in the coating method described below, it is preferable to select a solvent that does not dissolve the underlying layer. From this point of view, it is particularly preferable to contain alcohols.

The amount ratio of the solvent used in the present protective layer-forming coating liquid to the solid content differs depending on the coating method of the protective layer-forming coating liquid, and may be changed and used as appropriate so as to form a uniform coating film in the coating method to be applied.

### [Coating Method]

The method for coating a coating liquid for forming the present protective layer is not particularly limited, and examples thereof include a spray coating method, a spiral coating method, a ring coating method, and an immersion coating method.

The above coating method is performed to form a coating film, and the coating film is then dried. The temperature and time of drying may be any as long as necessary and sufficient drying can be achieved. However, if the protective layer is formed only by air-drying after the photosensitive layer coating, it is preferable to perform sufficient drying by the method described in the method for forming a photosensitive layer described below.

### [Method for Curing Present Protective Layer]

The present protective layer can be formed by coating the present protective layer-forming coating liquid, and then curing the coating liquid by applying external energy. Examples of the external energy used in the curing include heat, light, and radiation.

Examples of the method for applying heat energy include heating methods using gases such as air and nitrogen, steam, various heat media, infrared rays, and electromagnetic waves. The heating can be applied from the coated surface side or the support side. The heating temperature is preferably 100° C. or higher and 170° C. or lower.

As for the light energy, an ultraviolet (UV) irradiation light source having a light emission wavelength mainly in UV light, such as a high-pressure mercury lamp, a metal halide lamp, an electrodeless lamp bulb, or a light emitting diode, can be used. In addition, a visible light source can also be selected according to an absorption wavelength of the chain polymerizable compound or the photopolymerization initiator.

The amount of light irradiation is preferably 10 J/cm² or more, more preferably 30 J/cm² or more, and particularly preferably 100 J/cm² or more, from the viewpoint of curability. In addition, it is preferably 500 J/cm² or less, more preferably 300 J/cm² or less, and particularly preferably 200 J/cm² or less, from the viewpoint of electrical properties.

Examples of the radiation energy include those using an electron beam (EB).

Among these energies, those using the light energy are preferred from the viewpoint ease of reaction rate control, simplicity of apparatus, and length of pot life.

After the protective layer is cured, a heating step may be added from the viewpoint of alleviating residual stress, alleviating residual radicals, and improving electrical properties. The heating temperature is preferably 60° C. or higher, more preferably 100° C. or higher; and preferably 200° C. or lower, more preferably 150° C. or lower.

### (Layer Thickness)

The thickness of the present protective layer is preferably 0.5 μm or more, more preferably 1 μm or more, from the viewpoint of abrasion resistance. In addition, it is preferably 5 μm or less, more preferably 3 μm or less, from the viewpoint of electrical properties.

From the same viewpoint, the thickness of the present protective layer is preferably 1/50 or more relative to the thickness of the present photosensitive layer, more preferably 1/40 or more, and even more preferably 1/30 or more. In addition, it is preferably 1/5 or less, more preferably 1/10 or less, and even more preferably 1/20 or less.

### <Present Photosensitive Layer>

The photosensitive layer (also referred to as “the present photosensitive layer”) in the present electrophotographic photoconductor may be a layer containing at least a charge generation material (CGM) and a charge transport material.

The present photosensitive layer may be a single-layered photosensitive layer containing a charge generation material and a charge transport material in the same layer, or a multi-layered photosensitive layer in which a charge generation layer and a charge transport layer are separated.

### <Single-Layered Photosensitive Layer>

The present photosensitive layer, when it is a single-layered photosensitive layer, preferably contains at least a charge generation material (CGM), a hole transport material (HTM), an electron transport material (ETM), and a binder resin in the same layer.

### (Charge Generation Material)

Examples of the charge generation material used in the present photosensitive layer include various photoconductive materials such as inorganic photoconductive materials and organic pigments. Among them, organic pigments are particularly preferred, and a phthalocyanine pigment and an azo pigment are more preferred.

Specifically, when a phthalocyanine pigment is used as the charge generation material, metal-free phthalocyanines and phthalocyanines coordinated with a metal such as copper, indium, gallium, tin, titanium, zinc, vanadium, silicon or germanium, or an oxide or halide thereof, are used. Among them, X-type and t-type metal-free phthalocyanines, A-type, B-type, and D-type titanyl phthalocyanines, vanadyl phthalocyanines, chloroindium phthalocyanines, chlorogallium phthalocyanines, and hydroxygallium phthalocyanines, which have high sensitivity, are particularly suitable.

When an azo pigment is used, various known bisazo pigments and trisazo pigments are suitably used.

The charge generation material may be used alone or in any combination of two or more types in any ratio. When two or more charge generation materials are used together, the charge generation materials to be used together may each be mixed afterwards, or they may be mixed together during the production and processing steps of the charge generation materials, such as synthesis, pigmentation, and crystallization.

The charge generation material preferably has a small particle diameter from the viewpoint of electrical properties. Specifically, the particle diameter of the charge generation material is preferably 1 μm or less, more preferably 0.5 μm or less. The lower limit thereof is 0.01 μm or more. The particle diameter of the charge generation material refers to a particle diameter in the state contained in the photosensitive layer.

The amount of the charge generation material in the single-layered photosensitive layer is preferably 0.1% by mass or more, more preferably 0.5% by mass or more, from the viewpoint of sensitivity. In addition, it is preferably 50% by mass or less, more preferably 20% by mass or less, from the viewpoint of sensitivity and chargeability.

### (Charge Transport Material)

The charge transport material is classified into a hole transport material mainly having hole transport capability and an electron transport material mainly having electron transport capability. The present photosensitive layer, when it is a single-layered photosensitive layer, preferably contains at least a hole transport material and an electron transport material in the same layer.

### [Hole Transport Material]

The hole transport material (HTM) can be selected from known materials. Examples thereof include electron donating materials, such as heterocyclic compounds such as a carbazole derivative, an indole derivative, an imidazole derivative, an oxazole derivative, a pyrazole derivative, a thiadiazole derivative, and a benzofuran derivative, an aniline derivative, a hydrazone derivative, an arylamine derivative, a stilbene derivative, a butadiene derivative, and an enamine derivative, a compound obtained by bonding a plurality of these compounds, and a polymer having a group derived from these compounds in a main chain or a side chain.

Among them, a carbazole derivative, an arylamine derivative, a stilbene derivative, a butadiene derivative, an enamine derivative, and a compound obtained by bonding a plurality of these compounds are preferred, and an arylamine derivative and an enamine derivative are more preferred.

The hole transport material may be used alone or in any combination of two or more types in any ratio.

### [Electron Transport Material]

The electron transport material (ETM) can be selected from known materials. Examples thereof include: electron attracting materials, such as aromatic nitro compounds such as 2,4,7-trinitrofluorenone, cyano compounds such as tetracyanoquinodimethane, and quinone compounds such as diphenoquinone; and known cyclic ketone compounds and perylene pigments (perylene derivatives). Among them, quinone compounds and perylene pigments (perylene derivatives) are preferred, and quinone compounds are more preferred, from the viewpoint of electrical properties.

Among the quinone compounds, diphenoquinone and dinaphthylquinone are preferred from the viewpoint of electrical properties. Among them, dinaphthylquinone is more preferred.

The electron transport material may be used alone or in any combination of two or more types in any ratio.

Preferred examples of the structure of the electron transport material are shown below.

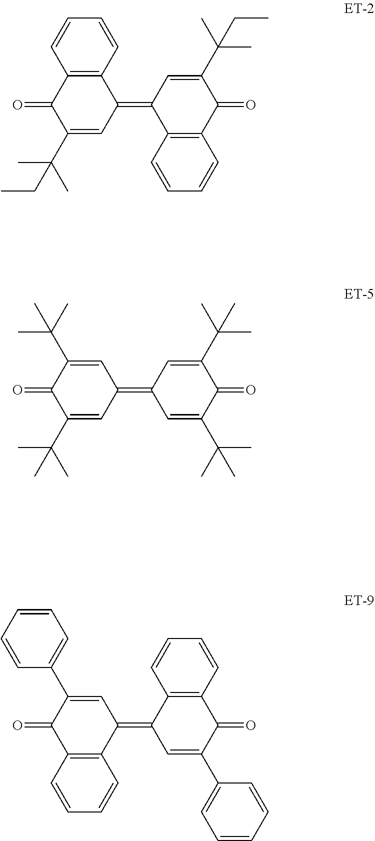 [SMILES: CCC(C)(C)C1=C/C(=C2/C=C(C(C)(C)CC)C(=O)c3ccccc32)c2ccccc2C1=O ; CC(C)(C)C1=CC(=C2C=C(C(C)(C)C)C(=O)C(C(C)(C)C)=C2)C=C(C(C)(C)C)C1=O ; O=C1C(c2ccccc2)=C/C(=C2/C=C(c3ccccc3)C(=O)c3ccccc32)c2ccccc21]

Among the above electron transport materials, ET-2 and ET-5 are preferred, and ET-2 is more preferred, from the viewpoint of electrical properties.

### (Binder Resin)

The following describes a binder resin used in the present photosensitive layer.

Examples of the binder resin used in the present photosensitive layer include: vinyl polymers such as polymethyl methacrylate, polystyrene, and polyvinyl chloride, or copolymers thereof; vinyl alcohol resins; polyvinyl butyral resins; polyvinyl formal resins; partially modified polyvinyl acetal resins; polyarylate resins; polyamide resins; polyurethane resins; polycarbonate resins; polyester resins; polyester carbonate resins; polyimide resins; phenoxy resins; epoxy resins; silicone resins; and partially-crosslinked cured product thereof. The above resins may each be modified with a silicon reagent or the like. These may be used alone or in any combination of two or more types in any ratio.

The binder resin used in the present photosensitive layer preferably contains one or two or more types of polymers obtained by interfacial polymerization.

As the binder resin obtained by interface polymerization, a polycarbonate resin and a polyester resin are preferred, and in particular, a polycarbonate resin or a polyarylate resin is preferred. In addition, a polymer obtained by using an aromatic diol as a raw material is particularly preferred.

### (Other Materials)

The present photosensitive layer may contain, in addition to the above-mentioned materials, known additives such as an antioxidant, a plasticizer, an ultraviolet absorber, an electron attracting compound, a leveling agent, and a visible light shielding agent, for improving film formability, flexibility, coating ability, contamination resistance, gas resistance, light resistance, and the like. The present photosensitive layer may also contain various additives such as a sensitizer, a dye, a pigment (excluding the charge generation material, the hole transport material, and the electron transport material described above), and a surfactant, as necessary. Examples of the surfactant include silicone oil and fluorine compounds. In the present invention, these may be appropriately used alone or in any combination of two or more types in any ratio.

For the purpose of reducing friction resistance on the surface of the photosensitive layer, the present photosensitive layer may contain a fluorine resin, a silicone resin, or the like, and may contain particles made of these resins or particles made of inorganic compounds such as aluminum oxide.

### (Layer Thickness)

When the present photosensitive layer is a single-layered photosensitive layer, the thickness of the present photosensitive layer is preferably 20 μm or more, more preferably 25 μm or more, from the viewpoint of dielectric breakdown resistance. In addition, it is preferably 50 μm or less, more preferably 40 μm or less, from the viewpoint of electrical properties.

### <Multi-Layered Photosensitive Layer>

When the present electrophotographic photoconductor has a multi-layered photosensitive layer, examples of the configuration thereof include a configuration in which a charge transport layer (CTL) containing a charge transport material is laminated on a charge generation layer (CGL) containing a charge generation material (CGM). In this case, the multi-layered photosensitive layer may also include any layer other than the charge generation layer (CGL) and the charge transport layer (CTL).

### <Charge Generation Layer (CGL)>

The charge generation layer generally contains a charge generation material (CGM) and a binder resin.

The charge generation material (CGM) and the binder resin are the same as in the single-layered photosensitive layer described above.

### (Other Components)

The charge generation layer may contain other components in addition to the charge generation material and the binder resin, as necessary. For example, the charge generation layer may contain known additives such as an antioxidant, a plasticizer, an ultraviolet absorber, an electron attracting compound, a leveling agent, a visible light shielding agent, and a filler, for the purpose of improving film formability, flexibility, coating ability, contamination resistance, gas resistance, light resistance, and the like.

### (Blending Ratio)

In the charge generation layer, if the ratio of the charge generation material is too high, the stability of the coating liquid may be decreased due to aggregation and the like of the charge generation material, while if the ratio of the charge generation material is too low, the sensitivity of the photoconductor may be decreased. Therefore, as for the blending ratio (mass) of the binder resin and the charge generation material, the charge generation material is preferably contained in an amount of 10 parts by mass or more, more preferably 30 parts by mass or more, relative to 100 parts by mass of the binder resin; and preferably 1,000 parts by mass or less, more preferably 500 parts by mass or less, even more preferably 300 parts by mass or less from the viewpoint of film strength, and still more preferably 200 parts by mass or less.

### (Layer Thickness)

The thickness of the charge generation layer is preferably 0.1 μm or more, more preferably 0.15 μm or more. In addition, it is preferably 10 μm or less, more preferably 0.6 μm or less.

### <Charge Transport Layer (CTL)>

The charge transport layer (CTL) generally contains a charge transport material and a binder resin.

The charge transport material and the binder resin are the same as in the single-layered photosensitive layer described above.

As for the blending ratio of the binder resin and the hole transport material (HTM) in the charge transport layer (CTL), the hole transport material (HTM) is preferably contained in an amount of 20 parts by mass or more, more preferably 30 parts by mass or more from the viewpoint of reducing residual potential, and even more preferably 40 parts by mass or more from the viewpoint of stability in repeated use and degree of charge transfer, relative to 100 parts by mass of the binder resin. In addition, the hole transport material (HTM) is preferably contained in an amount of 200 parts by mass or less from the viewpoint of thermal stability of the photosensitive layer, more preferably 150 parts by mass or less from the viewpoint of compatibility of the hole transport material (HTM) and the binder resin, and even more preferably 120 parts by mass or less from the viewpoint of glass transition temperature, relative to 100 parts by mass of the binder resin.

### (Other Components)

The charge transport layer may contain other components in addition to the electron transport material (ETM), the hole transport material (HTM), and the binder resin, as necessary. For example, the charge transport layer may contain known additives such as an antioxidant, a plasticizer, an ultraviolet absorber, an electron attracting compound, a leveling agent, a visible light shielding agent, and a filler, for the purpose of improving film formability, flexibility, coating ability, contamination resistance, gas resistance, light resistance, and the like.

### (Layer Thickness)

The thickness of the charge transport layer is not particularly limited. It is preferably 5 μm or more and 50 μm or less, more preferably 10 μm or more or 35 μm or less, and even more preferably 15 μm or more or 25 μm or less, from the viewpoint of electrical properties, image stability, and high image resolution.

### <Method for Forming Photosensitive Layer>

In both the multi-layer and single-layer types, each of the layers can be formed as follows.

Materials to be contained are dissolved or dispersed in a solvent to obtain a coating liquid, the coating liquid is coated on a conductive support by a known method, such as immersion coating, spray coating, nozzle coating, bar coating, roll coating, or blade coating, and the coating and drying steps are sequentially repeated for each layer, thereby forming layers.

However, it is not limited to this formation method.

The solvent or dispersion medium used for the preparation of the coating liquid is not particularly limited. Specific examples thereof include alcohols, ethers, aromatic hydrocarbons, and chlorinated hydrocarbons. These may be used alone or in any combination of two or more types in any type.

The amount of the solvent or dispersion medium used is not particularly limited. In view of the use purpose of each layer and properties of the solvent or dispersion medium selected, it is preferable to adjust the amount appropriately such that the physical properties of the coating liquid, such as solid content concentration and viscosity, are within the desired range.

As for the drying of the coating film, the coating film is preferably touch-dried at room temperature, and then heat-dried at a temperature range of generally 30° C. or higher and 200° C. or lower for 1 minute to 2 hours at a standstill or under air flow. The heating temperature may be constant, or the temperature may be varied during the heat drying.

### <Present Conductive Support>

The conductive support in the present electrophotographic photoconductor (also referred to as “the present conductive support”) is not particularly limited as long as it supports layers formed thereon and exhibits conductivity.

Examples of the present conductive support mainly used include: metal materials such as aluminum, aluminum alloy, stainless steel, copper, and nickel; resin materials with conductivity added by coexisting conductive powders of metal, carbon, and tin oxide; and resin, glass, and paper having conductive materials such as aluminum, nickel, and ITO (indium tin oxide alloy) vapor-deposited or coated on their surfaces.

The present conductive support used may be in the form of a drum, cylinder, sheet, belt, or the like.

The present conductive support may be a conductive support made of a metal material with a conductive material having a suitable resistance value coated thereon for controlling conductivity, surface properties, and the like, and for covering defects.

When a metal material such as an aluminum alloy is used as the present conductive support, the metal material may be coated with an anodic oxide film.

The average thickness of the anodic oxide film is preferably 20 μm or less, particularly preferably 7 μm or less.

The metal material, when coated with an anodic oxide film, is preferably subjected to a sealing treatment. The sealing treatment can be performed by a known method.

The surface of the present conductive support may be smooth or roughened by a special cutting method or polishing treatment. The surface may also be roughened by mixing particles having an appropriate particle diameter with the materials constituting the support.

In order to improve adhesion, blocking tendency, and the like, an undercoat layer described below may be provided between the present conductive support and the photosensitive layer.

### <Present Undercoat Layer>

The present electrophotographic photoconductor may include an undercoat layer (also referred to as “the present undercoat layer”) between the present photosensitive layer and the present conductive support.

Examples of the material used in the present undercoat layer include a resin and a resin having particles of organic pigments, metal oxides, or the like dispersed therein.

Examples of the organic pigments used in the undercoat layer include phthalocyanine pigments, azo pigments, and perylene pigments. Among them, phthalocyanine pigments and azo pigments, specifically the phthalocyanine pigment and the azo pigment as used as the charge generation material described above, can be exemplified.

Examples of the metal oxide particles used in the present undercoat layer include: metal oxide particles containing one metal element such as titanium oxide, aluminum oxide, silicon oxide, zirconium oxide, zinc oxide, or iron oxide; and metal oxide particles containing multiple metal elements such as calcium titanate, strontium titanate, and barium titanate. In the undercoat layer, only one type of particles may be used, or multiple types of particles may be used in mixture in any ratio and combination.

Among the above metal oxide particles, titanium oxide and aluminum oxide are preferred, and titanium oxide is particularly preferred.

The particle diameter of the metal oxide particles used in the present undercoat layer is not particularly limited. The average primary particle diameter is preferably 10 nm or more, and preferably 100 nm or less, more preferably 50 nm or less, from the viewpoint of properties of the undercoat layer and stability of the solution for forming the undercoat layer.

The binder resin used in the present undercoat layer can be selected and used, for example, from: polyvinyl acetal resins such as polyvinyl butyral resin; and insulating resins such as polyarylate resin, polycarbonate resin, polyester resin, phenoxy resin, acrylic resin, methacrylic resin, polyamide resin, polyurethane resin, epoxy resin, silicone resin, polyvinyl alcohol resin, and styrene-alkyd resin. However, it is not limited to these polymers. These binder resins may be used alone or in a mixture of two or more types, or may be used in a cured form with a curing agent.

Among them, polyvinyl acetal resins, alcohol-soluble copolymerized polyamides, modified polyamides, and the like are preferred because of their good dispersibility and coating ability. Among them, alcohol-soluble copolymerized polyamides are particularly preferred.

The mixing ratio of the particles to the binder resin can be arbitrarily selected. The ratio is preferably in a range of 10% by mass to 500% by mass from the viewpoint of stability and coating ability of the dispersion.

The film thickness of the present undercoat layer can be arbitrarily selected. The film thickness is preferably 0.1 μm or more, and is more preferably 20 μm or less, from the viewpoint of properties of the electrophotographic photoconductor and coating ability of the dispersion. The undercoat layer may contain a known antioxidant or the like.

### <Other Layers>

The present electrophotographic photoconductor may appropriately include other layers, as necessary, in addition to the present conductive support, the present photosensitive layer, the present protective layer, and the present undercoat layer described above.

### <<Present Image Formation Device>>

The present electrophotographic photoconductor can be used to configure an image formation device (also referred to as “the present image formation device”).

The present image formation device described below is an example of an image formation device that can be configured using the present electrophotographic photoconductor.

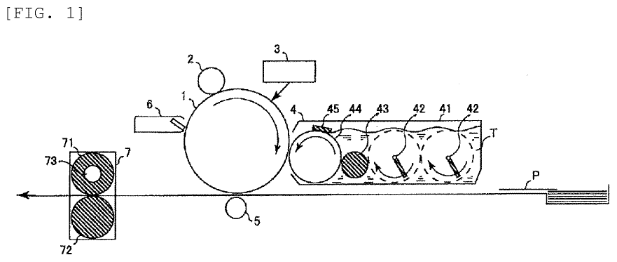

As shown in FIG. 1, the present image formation device includes a present electrophotographic photoconductor 1, a charging device 2, an exposing device 3, and a developing device 4, and may further include a transferring device 5, a cleaning device 6, and a fixing device 7, as necessary.

The present electrophotographic photoconductor 1 is not particularly limited as long as it is the present electrophotographic photoconductor described above. FIG. 1 shows, as one example thereof, a drum-shaped photoconductor including the photosensitive layer described above formed on the surface of a cylindrical conductive support. Along the outer peripheral surface of the present electrophotographic photoconductor 1, the charging device 2, the exposing device 3, the developing device 4, the transferring device 5, and the cleaning device 6 are disposed.

Examples of the charging device 2 include non-contact type corona charging devices such as corotron and scorotron, and contact type charging devices (direct type charging devices) that charge the photoconductor by bringing a voltage-applied charging member into contact with the surface of the photoconductor. Examples of the contact type charging devices include a charging roller and a charging brush. FIG. 1 shows a roller type charging device (charging roller) as one example of the charging device 2.

The type of the exposing device 3 is not particularly limited as long as the exposing device exposes the present electrophotographic photoconductor 1 to form an electrostatic latent image on the photosensitive surface of the present electrophotographic photoconductor 1.

The exposure may be performed by an internal exposure system of the photoconductor. The light used for exposure is arbitrary.

The type of a toner T is arbitrary, and may be a powder toner, a polymerized toner using a suspension polymerization method or an emulsion polymerization method, or the like.

The configuration of the developing device 4 is also arbitrary. The developing device 4 shown in FIG. 1 has a configuration in which the toner T is thinned by a regulating member (developing blade) 45, frictionally charged to a predetermined polarity, and brought into contact with the surface of the photoconductor 1 while being conveyed by being carried on a developing roller 44. However, it is not limited to this configuration.

The type of the transferring device 5 is not particularly limited, and may be a device using any method, such as an electrostatic transferring method such as corona transfer, roller transfer, or belt transfer, a pressure transferring method, or an adhesive transferring method.

The cleaning device 6 is not particularly limited. For example, any cleaning device such as a brush cleaner, a magnetic roller cleaner, or a blade cleaner can be used. If there is little or almost no toner remaining on the surface of the photoconductor, no cleaning device 6 may be used.

The configuration of the fixing device 7 is also arbitrary.

The image formation device may have a configuration capable of performing, for example, a static elimination step, in addition to the configurations described above.

In addition, the image formation device may have a configuration with further modifications, such as a configuration capable of performing steps such as a pre-exposure step and an auxiliary charging step, a configuration capable of performing offset printing, or a configuration of a full color tandem system using multiple toners.

### <<Present Electrophotographic Photoconductor Cartridge>>

The present electrophotographic photoconductor 1 can be used in combination with one or two or more of the charging device 2, the exposing device 3, the developing device 4, the transferring device 5, the cleaning device 6, and the fixing device 7, to thereby configure an integrated type cartridge (referred to as “the present electrophotographic photoconductor cartridge”).

The present electrophotographic photoconductor cartridge described below is an example of an electrophotographic photoconductor cartridge that can be configured using the present electrophotographic photoconductor.

The present electrophotographic photoconductor cartridge may have a configuration capable of being attached to or detached from an electrophotographic device main body, such as a copier or a laser beam printer. In this case, for example, when the present electrophotographic photoconductor 1 or other members are deteriorated, the image formation device is capable of detaching the electrophotographic photoconductor cartridge from the image formation device main body and attaching another new electrophotographic photoconductor cartridge thereto, thereby facilitating maintenance and management of the image formation device.

### <<Explanation of Terms and Phrases>>

In the present invention, the expression “from X to Y” (wherein X and Y each represent an arbitrary numeral) encompasses the meaning “X or more and Y or less” and also encompasses the meaning “preferably more than X” or “preferably less than Y”, unless otherwise indicated.

The expression “X or more” (wherein X represents an arbitrary numeral) or “Y or less” (wherein Y represents an arbitrary numeral) encompasses the meaning “preferably more than X” or “preferably less than Y”.

In the present specification, the term “and/or” means both “or” and “and”.

### EXAMPLES

Embodiments of the present invention are further specifically described below with reference to Examples. However, the following Examples are shown for the purpose of explaining the present invention in detail and the present invention is not to be limited to the following Examples without departing from the gist thereof, and may be implemented with any modification. The term “parts” in the following Examples and Comparative Examples refers to “parts by mass” unless otherwise specified.

In the present specification, the term DMF means N, N-dimethylformamide and the term MEHQ means 4-methoxyphenol.

### <Synthesis of Electron Transporting Compounds>

The synthesis methods of compounds 1 to 5 and comparative compounds 1 to 4 as the electron transporting compound are described below.

### Synthesis of Compound 1

The following describes a synthesis scheme of a compound 1.

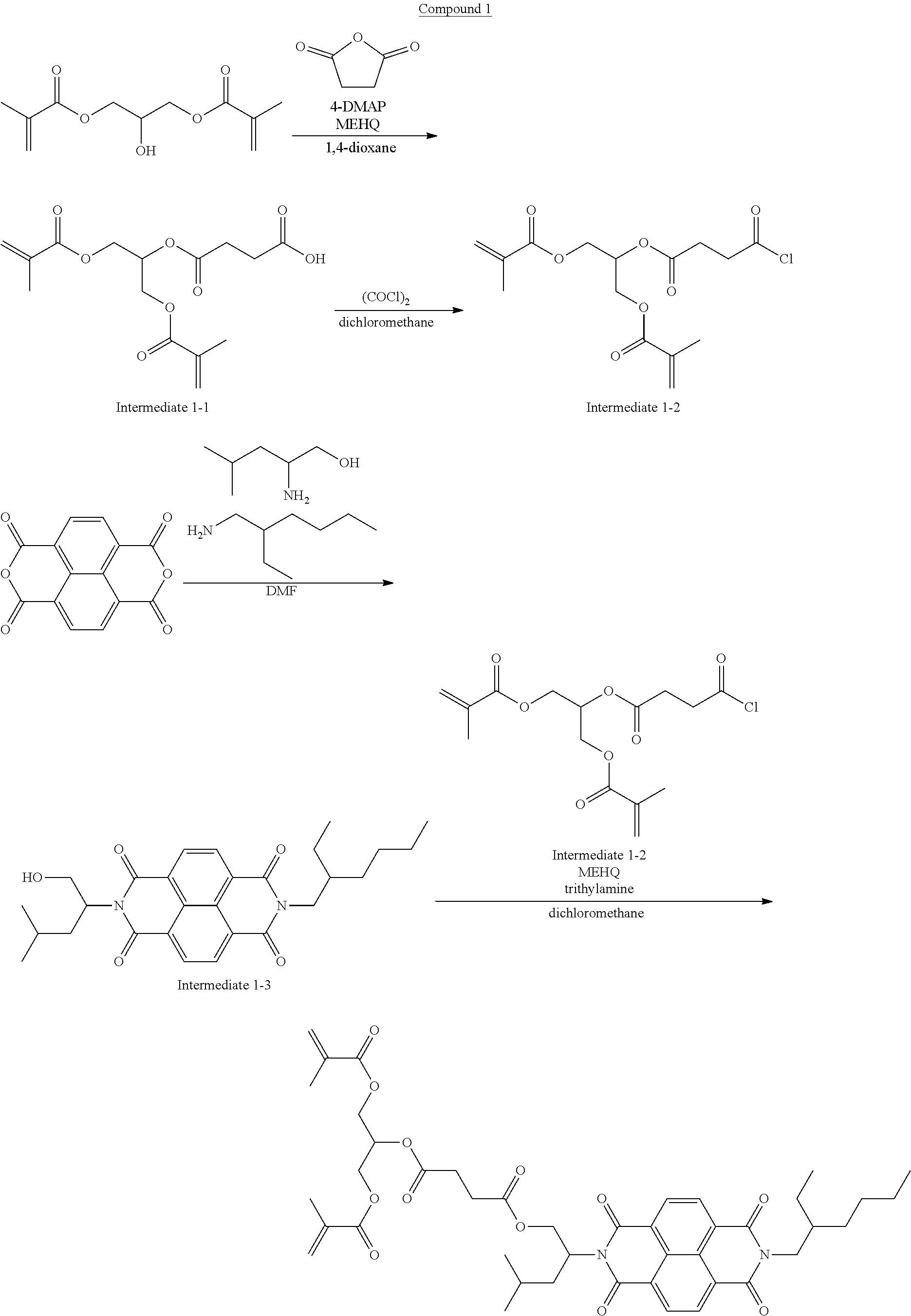

The following describes a synthesis procedure of the compound 1.

### Synthesis of Intermediate 1-1

Under a nitrogen atmosphere, 100 mL of 1,4-dioxane was added to a mixture of succinic anhydride (11.0 g, 109.5 mmol) and 4-DMAP (4-dimethylaminopyridine, 0.26 g, 2.19 mmol) to prepare a solution. To the solution, a solution of glycerol dimethacrylate (25 g, 109.5 mmol) dissolved in 50 mL of 1,4-dioxane and MEHQ (27 mg, 0.22 mmol) was added dropwise, and the resulting solution was stirred at 80° C. for 9 hours. After being cooled to room temperature, the resulting solution was poured into 200 ml of water and extracted with dichloromethane, and the organic layer was washed with water and then dried over magnesium sulfate. The solid was filtered, the solvent in the filtrate was distilled off under reduced pressure, and the residue was dried to obtain an intermediate 1-1 (yield: 30 g, yield rate: 83% by mass).

### Synthesis of Intermediate 1-2

Under a nitrogen atmosphere, 100 mL of dehydrated dichloromethane and 1 mL of dehydrated dimethylformamide were added to the intermediate 1-1 (21.6 g, 65.8 mmol) to prepare a solution, and the solution was ice-cooled. Oxalyl chloride (11.2 mL, 131.6 mmol) was added dropwise thereto, and the resulting solution was stirred under ice cooling for 2 hours and then stirred at room temperature for 12 hours. The solvent was distilled off under reduced pressure, and the residue was dried to obtain an intermediate 1-2 (yield: 21.5 g, yield rate: 94% by mass).

### Synthesis of Intermediate 1-3

Under a nitrogen atmosphere, 60 mL of N, N-dimethylformamide was added to naphthalene-1,4,5,8-tetracarboxylic dianhydride (7.18 g, 26.8 mmol) to prepare a solution. To the solution, a solution obtained by mixing L-(+)-leucinol (4.71 g, 40.2 mmol) dissolved in 40 mL of N, N-dimethylformamide with 2-ethylhexylamine (5.19 g, 40.2 mmol) was added dropwise, and the resulting solution was stirred at 120° C. for 8 hours. After being cooled to room temperature, the resulting solution was poured into 200 mL of ice water, and 1N hydrochloric acid was added thereto to acidify the reaction solution. After extraction with dichloromethane, the organic layer was washed with water and then dried over magnesium sulfate, and the resulting solid was filtered. The solvent in the filtrate was distilled off under reduced pressure, and the residue was dried to obtain an intermediate 1-3 (yield: 10.7 g, yield rate: 84% by mass).

### Synthesis of Compound 1

Under a nitrogen atmosphere, 150 mL of dehydrated dichloromethane and triethylamine (11.7 mL, 84.4 mmol) were added to the intermediate 1-3 (10.1 g, 21.1 mmol) and 4-methoxyphenol (0.01 g) to prepare a solution, and the solution was ice-cooled. The intermediate 1-2 (14.6 g, 42.2 mmol) dissolved in 50 mL of dehydrated dichloromethane was added dropwise thereto, and the resulting solution was stirred under ice cooling for 1 hour and then stirred at room temperature for 12 hours. The reaction solution was poured into 100 ml of water and extracted with dichloromethane, and the organic layer was washed with water and then dried over magnesium sulfate. The resulting solid was filtered, the solvent in the filtrate was distilled off under reduced pressure, and the residue was subjected to silica gel column chromatography to obtain a compound 1 (yield: 5.3 g, yield rate: 32% by mass).

### Synthesis of Compound 2

The following describes a synthesis scheme of a compound 2. The compound 2 is a mixture of a compound 2-a and a compound 2-b.

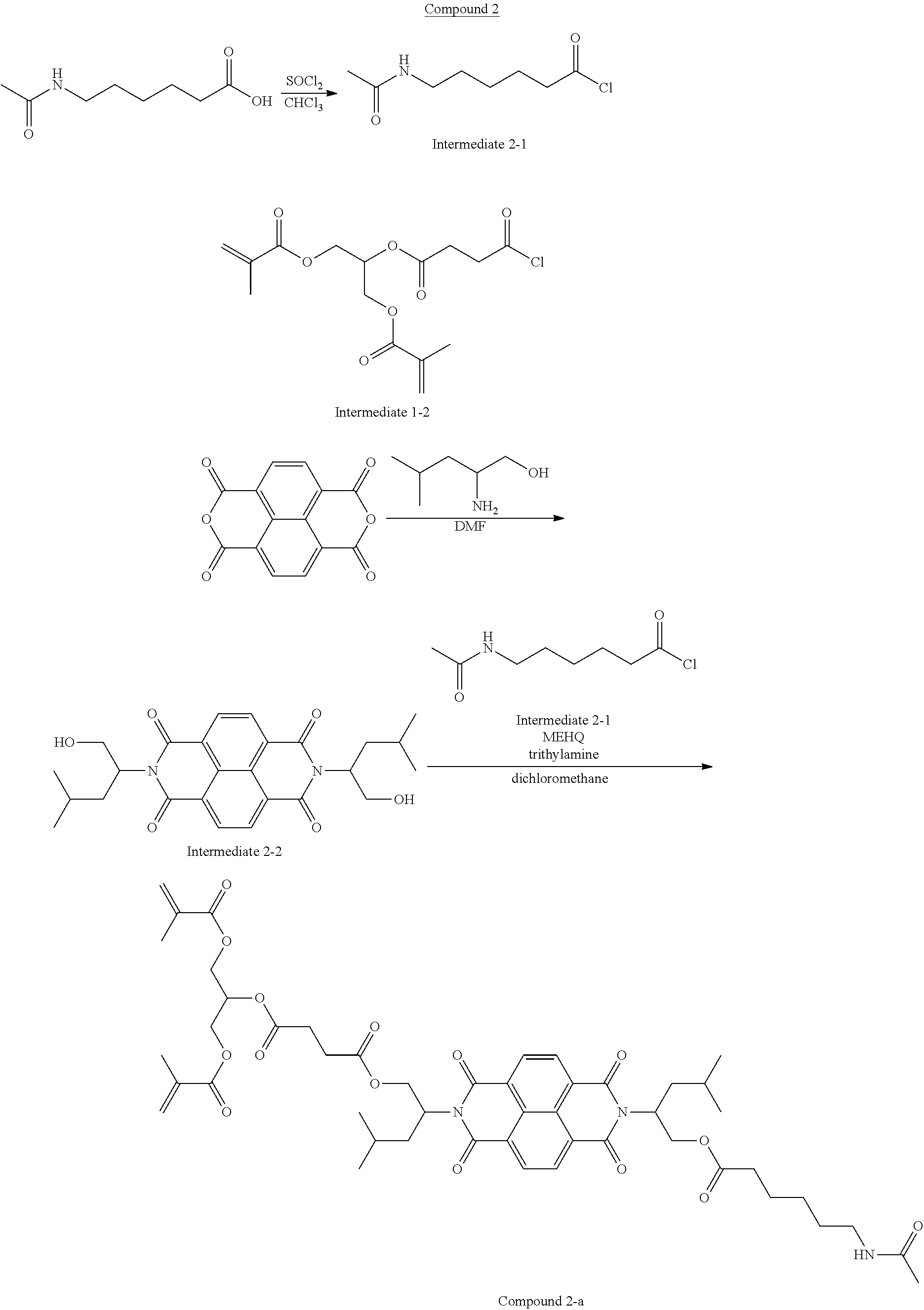

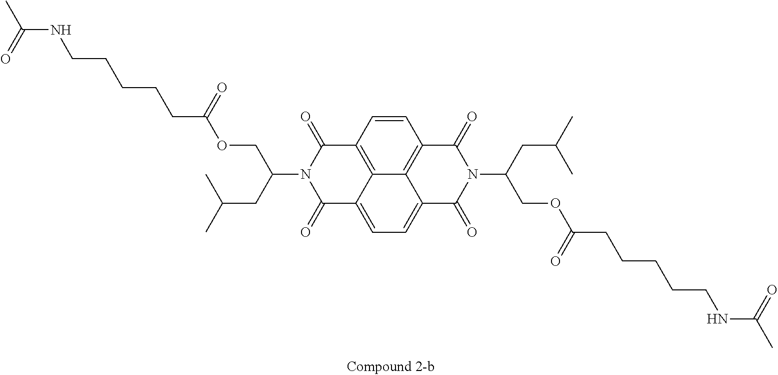 [SMILES: CC(=O)NCCCCCC(=O)OCC(CC(C)C)N1C(=O)c2ccc3c4c(ccc(c24)C1=O)C(=O)N(C(COC(=O)CCCCCNC(C)=O)CC(C)C)C3=O]

The following describes a synthesis procedure of the compound 2.

### Synthesis of Intermediate 2-1

Under a nitrogen atmosphere, 60 mL of dehydrated chloroform was added to 6-acetamidohexanoic acid (7.8 g, 45.0 mmol) to prepare a solution, and the solution was ice-cooled. To the solution, thionyl chloride (3.3 mL, 45.0 mmol) was added dropwise, and the resulting solution was stirred under ice cooling for 30 minutes and then stirred at 60° C. for 1 hour. After being cooled to room temperature, the solvent was distilled off under reduced pressure, and the residue was dried to obtain an intermediate 2-1 (yield: 8.5 g, yield rate: 98% by mass).

### Synthesis of Intermediate 2-2

Under a nitrogen atmosphere, 150 mL of N, N-dimethylformamide was added to naphthalene-1,4,5,8-tetracarboxylic dianhydride (4.5 g, 16.9 mmol) and L-(+)-leucinol (4.4 mL, 33.7 mmol) to prepare a solution, and the solution was stirred at 150° C. for 6 hours. After being cooled to room temperature, the solution was poured into 200 mL of ice water, and 1N hydrochloric acid was added thereto to acidify the solution. After extraction with ethyl acetate, the organic layer was washed with water and then dried over magnesium sulfate, and the resulting solid was filtered. The solvent in the filtrate was distilled off under reduced pressure, and the residue was dried to obtain an intermediate 2-2 (yield: 7.8 g, yield rate: 99% by mass).

### Synthesis of Compound 2

Under a nitrogen atmosphere, 100 mL of dehydrated dichloromethane and triethylamine (8.9 mL, 64.2 mmol) were added to the intermediate 2-2 (5.0 g, 10.7 mmol) and 4-methoxyphenol (0.02 g) to prepare a solution, and the solution was ice-cooled. The intermediate 1-2 (5.6 g, 16.1 mmol) and the intermediate 2-1 (3.1 g, 16.1 mmol) dissolved in 100 mL of dehydrated dichloromethane were added dropwise thereto, and the resulting solution was stirred under ice cooling for 1 hour and then stirred at room temperature for 12 hours. The solvent in the reaction solution was distilled off under reduced pressure, and the residue was subjected to silica gel column chromatography to obtain a compound 2 (yield: 9.0 g, yield rate: 90% by mass, molar ratio of compound 2-a and compound 2-b (2-a: 2-b)=2:1).

### Synthesis of Compound 3

The following describes a synthesis scheme of a compound 3. The compound 3 is a mixture of a compound 3-a and a compound 3-b.

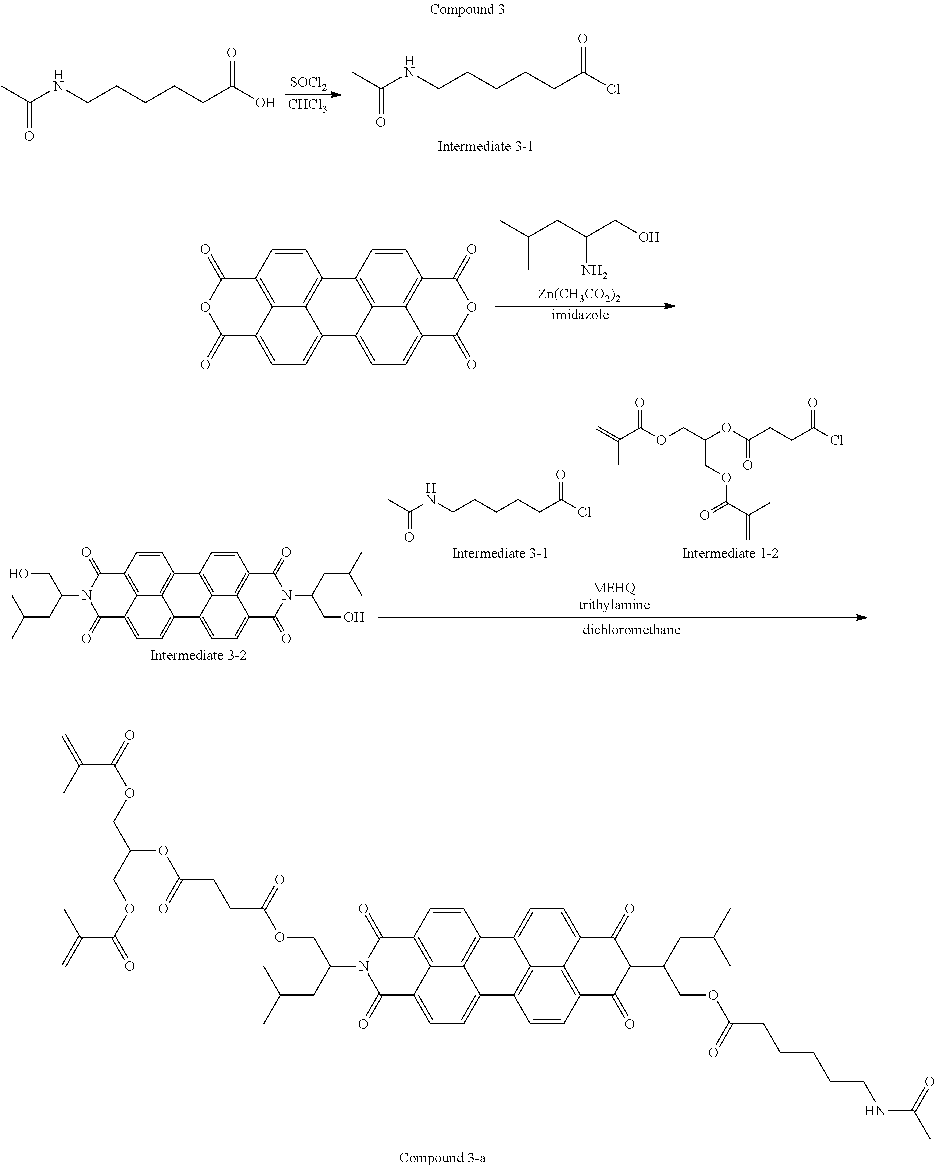

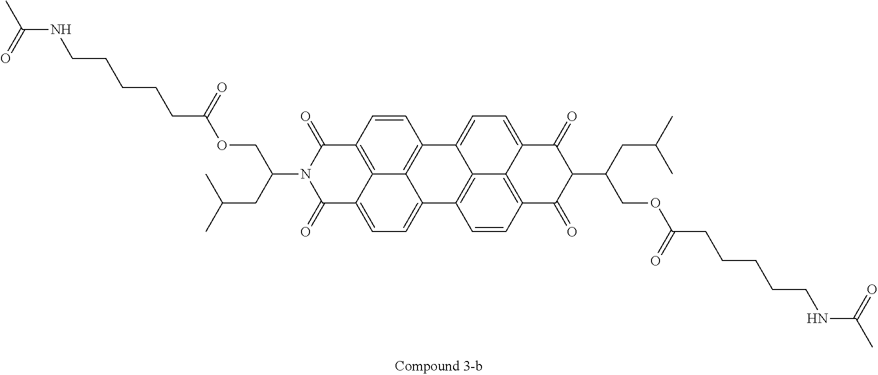 [SMILES: CC(=O)NCCCCCC(=O)OCC(CC(C)C)C1C(=O)c2ccc3c4ccc5c6c(ccc(c7ccc(c2c37)C1=O)c64)C(=O)N(C(COC(=O)CCCCCNC(C)=O)CC(C)C)C5=O]

The following describes a synthesis procedure of the compound 3.

### Synthesis of Intermediate 3-1

Under a nitrogen atmosphere, 60 mL of dehydrated chloroform was added to 6-acetamidohexanoic acid (7.8 g, 45.0 mmol) to prepare a solution, and the solution was ice-cooled. To the solution, thionyl chloride (3.3 mL, 45.0 mmol) was added dropwise, and the resulting solution was stirred under ice cooling for 30 minutes and then stirred at 60° C. for 1 hour. After being cooled to room temperature, the solvent was distilled off under reduced pressure, and the residue was dried to obtain an intermediate 3-1 (yield: 8.5 g, yield rate: 98% by mass).

### Synthesis of Intermediate 3-2

Under a nitrogen atmosphere, 3,4,9,10-perylenetetracarboxylic dianhydride (11.4 g, 29.0 mmol), zinc acetate (5.32 g, 29.0 mmol), 50 g of imidazole, and L-(+)-leucinol (8.5 g, 72.5 mmol) were mixed, and the mixture was stirred at 160° C. for 7 hours. After being cooled to room temperature, the mixture was dissolved in dichloromethane to prepare a solution, the solution was poured into 200 mL of ice water, and 1N hydrochloric acid was added thereto to acidify the reaction solution. After extraction with dichloromethane, the organic layer was washed with water and then dried over magnesium sulfate. The resulting solid was filtered, the solvent in the filtrate was distilled off under reduced pressure, and the residue was subjected to silica gel column chromatography to obtain an intermediate 3-2 (yield: 12.0 g, yield rate: 70% by mass).

### Synthesis of Compound 3

Under a nitrogen atmosphere, 100 mL of dehydrated dichloromethane and triethylamine (13.7 mL, 99.0 mmol) were added to the intermediate 3-2 (9.7 g, 16.5 mmol) and 4-methoxyphenol (0.01 g) to prepare a solution, and the solution was ice-cooled. The intermediate 3-1 (4.7 g, 24.7 mmol) and the intermediate 1-2 (8.7 g, 24.7 mmol) dissolved in 80 mL of dehydrated dichloromethane were added dropwise thereto, and the resulting solution was stirred under ice cooling for 1 hour and then stirred at room temperature for 12 hours. The solvent in the reaction solution was distilled off under reduced pressure, and the residue was subjected to silica gel column chromatography to obtain a compound 3 (yield: 15.5 g, yield rate: 89% by mass, molar ratio of compound 3-a and compound 3-b (3-a: 3-b)=2:1).

### Synthesis of Compound 4

The following describes a synthesis scheme of a compound 4. The compound 4 is a mixture of a compound 4-a and a compound 4-b.

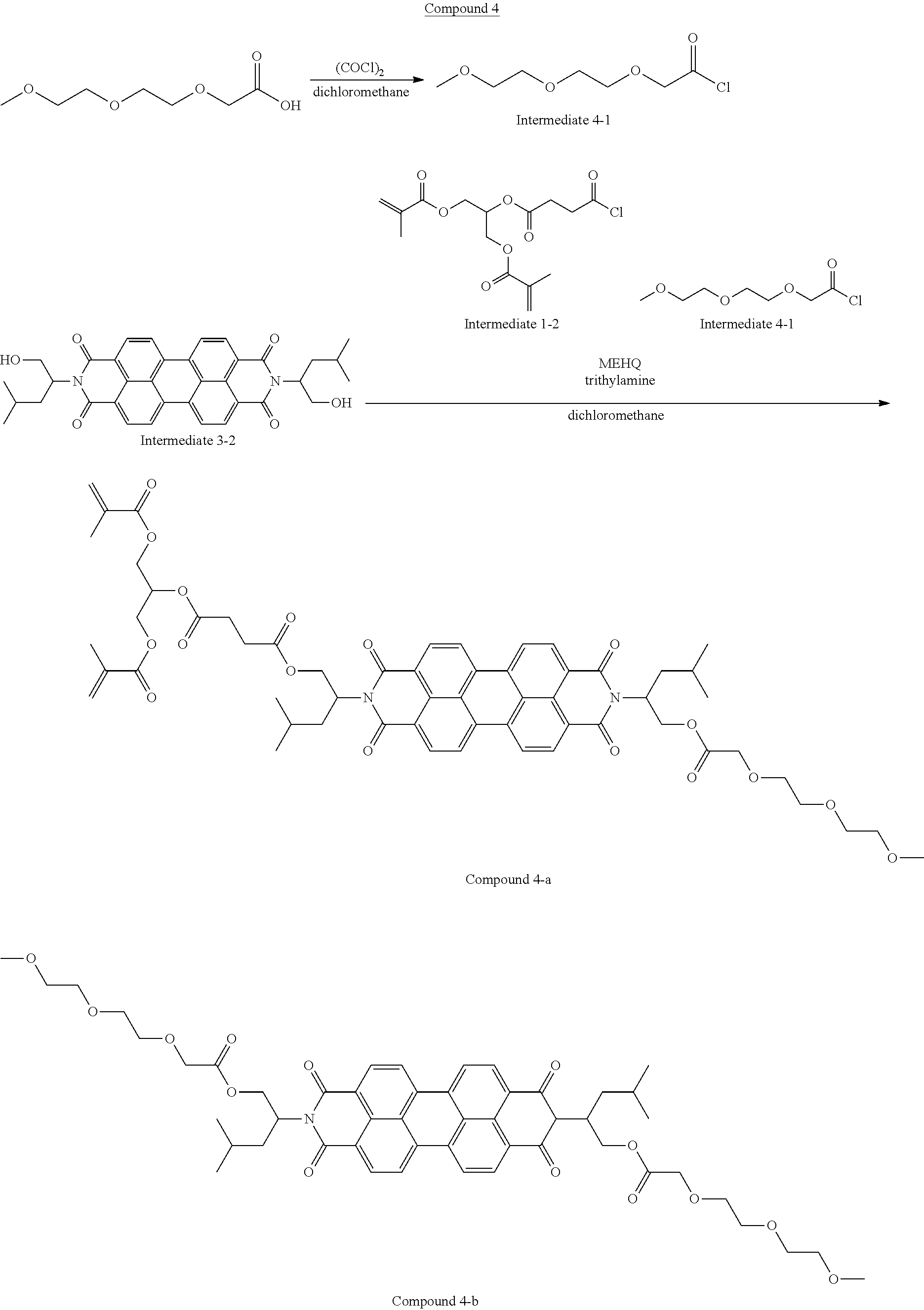

The following describes a synthesis procedure of the compound 4.

### Synthesis of Intermediate 4-1

Under a nitrogen atmosphere, 100 mL of dehydrated dichloromethane was added to [2-(2-methoxyethoxy)ethoxy]acetic acid (6.3 g, 35.3 mmol) to prepare a solution, and the solution was ice-cooled. To the solution, 0.5 mL of N, N-dimethylformamide was added, then oxalyl chloride (6.1 mL, 70.7 mmol) was added dropwise, and the resulting solution was stirred under ice cooling for 1 hour and then stirred at room temperature for 12 hours. The solvent was distilled off under reduced pressure, and the residue was dried to obtain an intermediate 4-1 (yield: 6.9 g, yield rate: 99% by mass).

### Synthesis of Compound 4

Under a nitrogen atmosphere, 200 mL of dehydrated dichloromethane and triethylamine (19.5 mL, 141 mmol) were added to the intermediate 3-2 (13.9 g, 23.5 mmol) and 4-methoxyphenol (0.04 g) to prepare a solution, and the solution was ice-cooled. The intermediate 4-1 (12.2 g, 35.3 mmol) and the intermediate 1-2 (6.94 g, 35.3 mmol) dissolved in 80 mL of dehydrated dichloromethane were added dropwise thereto, and the resulting solution was stirred under ice cooling for 1 hour and then stirred at room temperature for 12 hours. The solvent in the reaction solution was distilled off under reduced pressure, and the residue was subjected to silica gel column chromatography to obtain a compound 4 (yield: 22 g, yield rate: 88% by mass, molar ratio of compound 4-a and compound 4-b (4-a: 4-b)=2:1).

### Synthesis of Compound 5

The following describes a synthesis scheme of a compound 5.

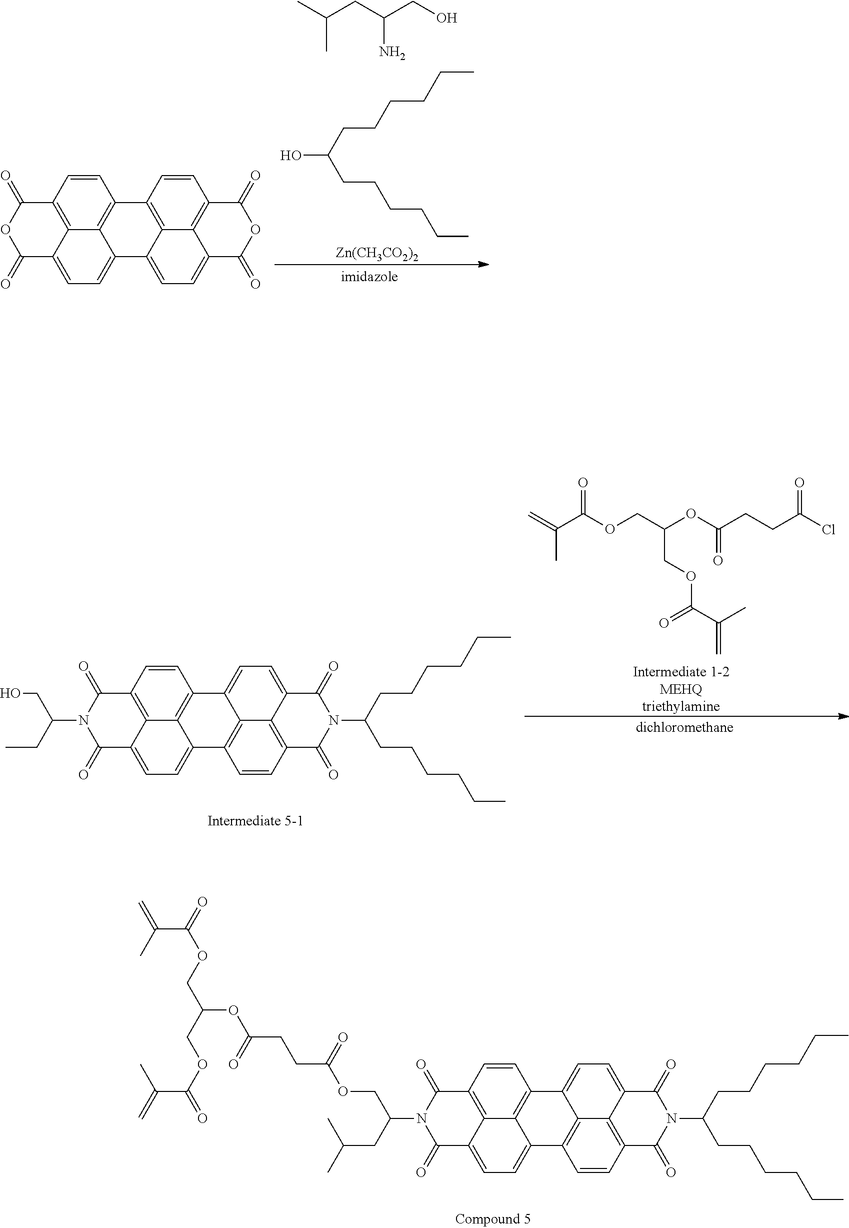

The following describes a synthesis procedure of the compound 5.

### Synthesis of Intermediate 5-1

Under a nitrogen atmosphere, 3,4,9,10-perylenetetracarboxylic dianhydride (5.26 g, 13.4 mmol), zinc acetate (2.46 g, 13.4 mmol), 50 g of imidazole, L-(+)-leucinol (2.40 g, 20.1 mmol), and tridecane-7-amine (4.00 g, 20.1 mmol) were mixed, and the mixture was stirred at 160° C. for 8 hours. After being cooled to room temperature, the mixture was dissolved in dichloromethane to prepare a solution, the solution was poured into 200 mL of ice water, and 1N hydrochloric acid was added thereto to acidify the reaction solution. After extraction with dichloromethane, the organic layer was washed with water and then dried over magnesium sulfate. The resulting solid was filtered, and the solvent in the filtrate was distilled off under reduced pressure. The residue was washed with methanol and then dried to obtain an intermediate 5-1 (yield: 5.38 g, yield rate: 60% by mass).

### Synthesis of Compound 5

Under a nitrogen atmosphere, 100 mL of dehydrated dichloromethane and triethylamine (4.4 mL, 31.9 mmol) were added to the intermediate 5-1 (5.38 g, 7.99 mmol) and 4-methoxyphenol (0.02 g) to prepare a solution, and the solution was ice-cooled. The intermediate 1-2 (5.55 g, 16.0 mmol) dissolved in 10 mL of dehydrated dichloromethane was added dropwise thereto, and the resulting solution was stirred under ice cooling for 2 hours and then stirred at room temperature for 12 hours. The reaction solution was poured into 100 mL of water and extracted with dichloromethane, and the organic layer was washed with water and then dried over magnesium sulfate. The resulting solid was filtered, the solvent in the filtrate was distilled off under reduced pressure, and the residue was subjected to silica gel column chromatography to obtain a compound 5 (yield: 2.2 g, yield rate: 28% by mass).

### Synthesis of Comparative Compound 1

The following describes a synthesis scheme of a comparative compound 1.

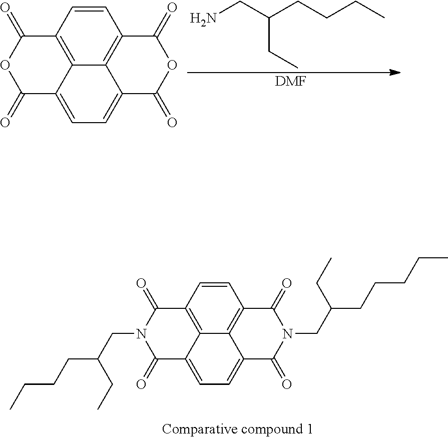

The following describes a synthesis procedure of the comparative compound 1.

### Synthesis of Comparative Compound 1

Under a nitrogen atmosphere, 100 mL of N, N-dimethylformamide was added to naphthalene-1,4,5,8-tetracarboxylic dianhydride (10.0 g, 37.3 mmol) to prepare a solution. To the solution, 2-ethylhexylamine (14.5 g, 111.9 mmol) was added dropwise, and the resulting solution was stirred at 120° C. for 8 hours. After being cooled to room temperature, the resulting solution was poured into 200 mL of ice water, and 1N hydrochloric acid was added thereto to acidify the reaction solution. After extraction with dichloromethane, the organic layer was washed with water and then dried over magnesium sulfate, and the resulting solid was filtered. The solvent in the filtrate was distilled off under reduced pressure, and the residue was dried to obtaining a comparative compound 1 (yield: 17.4 g, yield rate: 93% by mass).

### Synthesis of Comparative Compound 2

The following describes a synthesis scheme of a comparative compound 2.

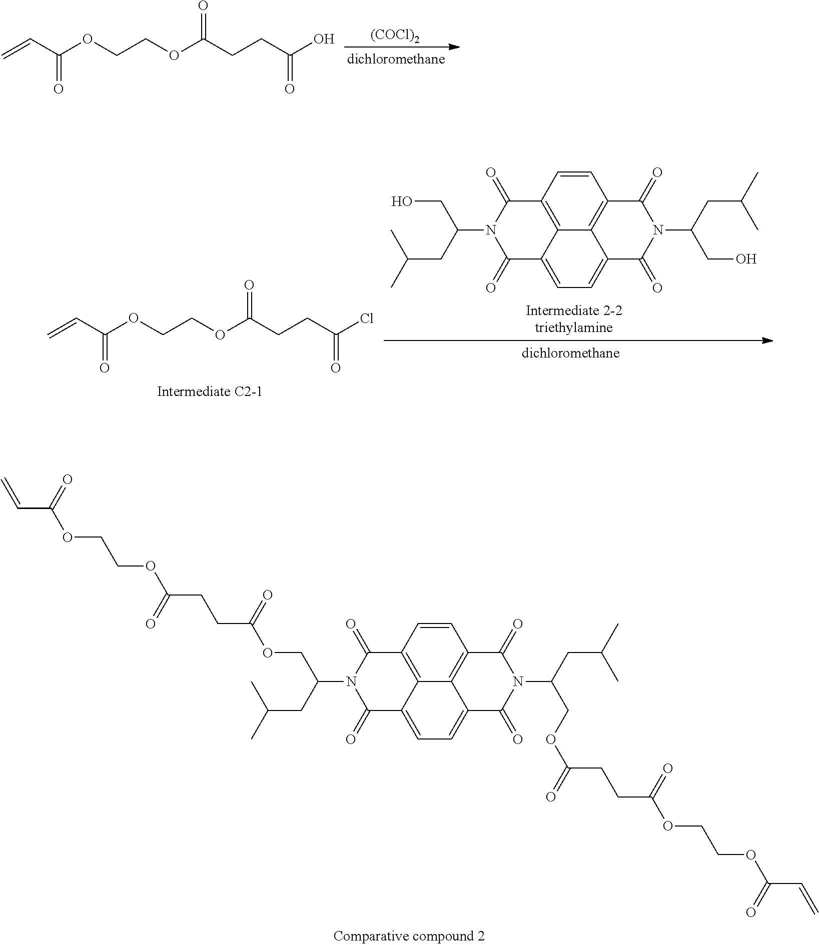

The following describes a synthesis procedure of the comparative compound 2.

### Synthesis of Intermediate C2-1

Under a nitrogen atmosphere, 200 mL of dehydrated dichloromethane was added to mono(2-acryloyloxyethyl) succinate (21.7 g, 100.4 mmol) to prepare a solution, and the solution was ice-cooled. Oxalyl chloride (11.2 mL, 130.5 mmol) was added dropwise thereto, and the resulting solution was stirred under ice cooling for 1 hour and then stirred at room temperature for 12 hours. The solvent in the reaction solution was distilled off under reduced pressure, and the residue was dried to obtain a compound C2-1 (yield: 23 g, yield rate: 97% by mass).

### Synthesis of Comparative Compound 2

Under a nitrogen atmosphere, 50 mL of dehydrated dichloromethane and triethylamine (5.1 mL, 36.8 mmol) were added to the intermediate 2-2 (4.3 g, 9.21 mmol) to prepare a solution, and the solution was ice-cooled. The intermediate C2-1 (4.8 g, 20.3 mmol) dissolved in 50 mL of dehydrated dichloromethane was added dropwise thereto, and the resulting solution was stirred under ice cooling for 1 hour and then stirred at room temperature for 1 hour. The reaction solution was poured into 100 mL of water and extracted with dichloromethane, and the organic layer was washed with water and then dried over magnesium sulfate. The resulting solid was filtered, the solvent in the filtrate was distilled off under reduced pressure, and the residue was subjected to silica gel column chromatography to obtain a comparative compound 2 (yield: 3.5 g, yield rate: 45% by mass).

### Synthesis of Comparative Compound 3

The following describes a synthesis scheme of a comparative compound 3.

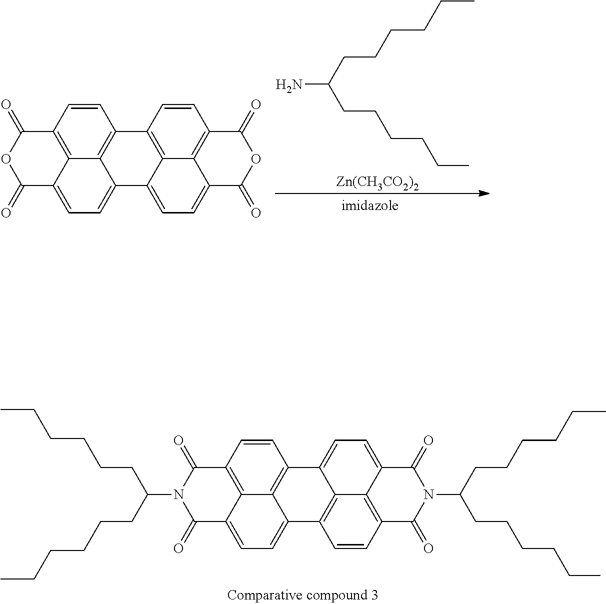 [SMILES: O=C1OC(=O)c2ccc3c4ccc5c6c(ccc(c7ccc1c2c73)c64)C(=O)OC5=O ; CCCCCCC(N)CCCCCC ; "Zn(CH3CO2)2" ; CCCCCCC(CCCCCC)N1C(=O)c2ccc3c4ccc5c6c(ccc(c7ccc(c2c37)C1=O)c64)C(=O)N(C(CCCCCC)CCCCCC)C5=O]

The following describes a synthesis procedure of the comparative compound 3.

### Synthesis of Comparative Compound 3

Under a nitrogen atmosphere, 3,4,9,10-perylenetetracarboxylic dianhydride (2.62 g, 6.69 mmol), zinc acetate (0.92 g, 5.02 mmol), 10 g of imidazole, and tridecane-7-amine (4.00 g, 20.1 mmol) were mixed, and the mixture was stirred at 160° C. for 5 hours. After being cooled to room temperature, the mixture was dissolved in dichloromethane to prepare a solution, the solution was poured into 200 mL of ice water, and 1N hydrochloric acid was added thereto to acidify the reaction solution. After extraction with dichloromethane, the organic layer was washed with water and then dried over magnesium sulfate. The resulting solid was filtered, the solvent in the filtrate was distilled off under reduced pressure, and the residue was subjected to silica gel column chromatography to obtain a comparative compound 3 (yield: 4.2 g, yield rate: 83% by mass).

### Synthesis of Comparative Compound 4

The following describes a synthesis scheme of a comparative compound 4.

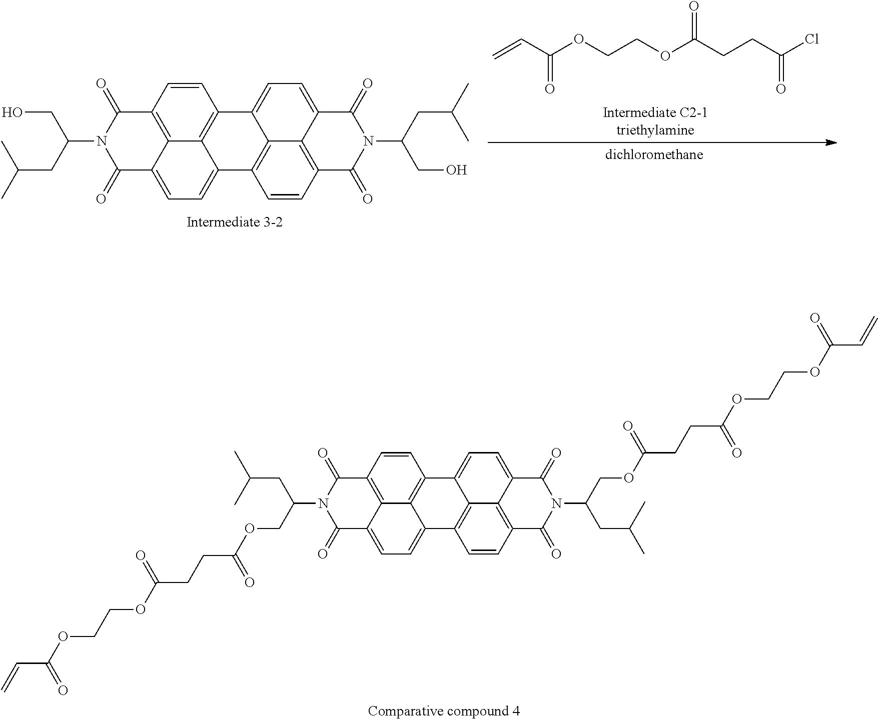

The following describes a synthesis procedure of the comparative compound 4.

### Synthesis of Comparative Compound 4

Under a nitrogen atmosphere, 100 mL of dehydrated dichloromethane and triethylamine (1.5 mL, 10.8 mmol) were added to the intermediate 3-2 (1.6 g, 2.71 mmol) to prepare a solution, and the solution was ice-cooled. The intermediate C2-1 (1.4 g, 5.96 mmol) dissolved in 10 mL of dehydrated dichloromethane was added dropwise thereto, and the resulting solution was stirred under ice cooling for 1 hour and then stirred at room temperature for 1 hour. The reaction solution was poured into 100 ml of water and extracted with dichloromethane, and the organic layer was washed with water and then dried over magnesium sulfate. The resulting solid was filtered, the solvent in the filtrate was distilled off under reduced pressure, and the residue was subjected to silica gel column chromatography to obtain a comparative compound 4 (yield: 2.3 g, yield rate: 85% by mass).

### <Evaluation of Solubility in Organic Solvent>

Eight parts by mass of each of the compounds 1 to 5 or the comparative compound 3 were added to 100 parts by mass of a mixed solvent of toluene/2-propanol in a mass ratio of 3/7 at room temperature (25° C.), and 8 parts by mass of each of the comparative compounds 1, 2, and 4 were added to 100 parts by mass of a mixed solvent of THF/2-propanol in a mass ratio of 3/7 at room temperature (25° C.); each mixture was stirred for 10 minutes using a rotor without heating or cooling; the state of dissolution was then visually observed; and the solubility was evaluated according to the following criteria.

If any undissolved residue was observed, the mixture was heated in a water bath at a constant temperature (45° C.) for 10 minutes, and the state of dissolution was again visually observed to evaluate the solubility as follows. The evaluation results are shown in Table 1.

⊚: Those that dissolve completely at room temperature
◯: Those that are observed to have undissolved residue at room temperature and dissolve completely after stirring at room temperature for 10 minutes or more, or after heating at 40° C. for less than 10 minutes
Δ: Those that dissolve completely after heating at 40° C. for 10 minutes or more
x: Those that are observed to have undissolved residue even after heating at 40° C. for 10 minutes or more

TABLE 1

| | Electron transporting | |
|---|---|---|
| | compound | Solubility |
| Example 1 | Compound 1 | ⊚ |
| Example 2 | Compound 2 | ⊚ |
| Example 3 | Compound 3 | ○ |
| Example 4 | Compound 4 | ○ |
| Example 5 | Compound 5 | ⊚ |
| Comparative | Comparative compound 1 | x |
| Example 1 | | |
| Comparative | Comparative compound 2 | Δ |
| Example 2 | | |
| Comparative | Comparative compound 3 | x |
| Example 3 | | |
| Comparative | Comparative compound 4 | x |
| Example 4 | | |

From the results in Table 1, it was confirmed that all of the compounds in Examples 1 to 5 exhibited superior solubility in organic solvents compared to those in Comparative Examples 1 to 4.

The electron transporting compounds in Examples 1 to 5 were each the compound represented by the formula (1). The compound is an electron transporting compound having a left-right asymmetric structure in which an electron transporting skeleton is centered, with a side chain having a polymerizable functional group on one side, and with a side chain having no polymerizable functional group on the other side.

From the results of the above Examples and Comparative Examples, as well as the results of the tests conducted by the present inventors to date, it is presumed that the compound having a left-right asymmetric structure in which an electron transporting skeleton is centered, with a side chain having a polymerizable functional group on one side, and with a side chain having no polymerizable functional group on the other side, since it has a left-right asymmetry and one of the sides is a side chain having no polymerizable functional group, has low crystallinity and good solubility in solvents, especially alcohols.

### <Production of Electrophotographic Photoconductor>

### (Preparation of Undercoat Layer-Forming Coating Liquid P1)

Twenty parts of D-type titanyl phthalocyanine showing a clear peak at a diffraction angle of 2θ=27.3°±0.2° in powder X-ray diffraction using Cukα rays, and 280 parts of 1,2-dimethoxyethane were mixed and ground for 2 hours using a sand grind mill for atomization dispersion treatment. To the mixture, 400 parts of a 1,2-dimethoxyethane solution containing 2.5% by mass of polyvinyl butyral (trade name “Denka butyral” #6000C, manufactured by Denka Company Limited) and 170 parts of 1,2-dimethoxyethane were further added and mixed, thereby preparing an undercoat layer-forming coating liquid P1 having a solid content concentration of 3.4% by mass.

### (Preparation of Single-Layered Photosensitive Layer-Forming Coating Liquid Q1)

To 793.35 parts of a mixed solvent of tetrahydrofuran (hereinafter appropriately abbreviated as THF) and toluene (hereinafter appropriately abbreviated as TL) (THF: 80% by mass, TL: 20% by mass), 2.6 parts of D-type titanyl phthalocyanine showing a clear peak at a diffraction angle of 2θ=27.3°+0.2° in powder X-ray diffraction using CuKα rays, 1.3 parts of a perylene pigment 1 having the following structure, 0.5 parts of a polyvinyl butyral resin, 100 parts of the following hole transport material (HTM48, molecular weight of 748), 60 parts of the following electron transport material (ET-2, molecular weight of 424.2), 100 parts of a polycarbonate resin having a biphenyl structure, and 0.05 parts of a silicone oil (tradename: KF-96, manufactured by Shin-Etsu Chemical Co., Ltd.) as a leveling agent were added and mixed, thereby preparing a single-layered photosensitive layer-forming coating liquid Q1 having a solid content concentration of 25% by mass.

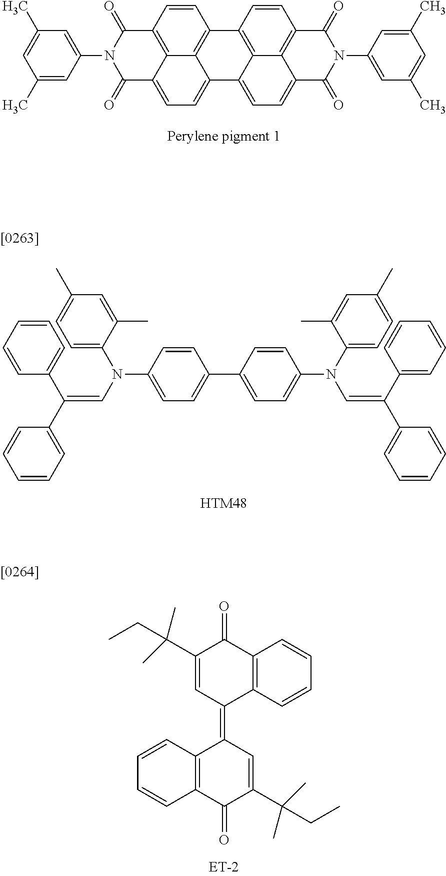

### (Preparation of Protective Layer-Forming Coating Liquid S1)

A curable compound (polyester acrylate: product name “Aronix M-9050”, manufactured by Toagosei Co., Ltd.) dissolved in a mixed solvent of toluene and 2-propanol, benzophenone and Omnirad TPO H (2,4,6-trimethylbenzoyl-diphenyl phosphine oxide) as polymerization initiators, and the compound 1 as an electron transporting compound were mixed to prepare a protective layer-forming coating liquid S1 (solid content concentration of approximately 8.0% by mass) in which the mass ratio of electron transporting compound/M-9050/benzophenone/Omnirad TPO H was 100/50/1/2 and the mass ratio of the solvent composition of toluene/2-propanol was 3/7.

### (Preparation of Protective Layer-Forming Coating Liquids S2 to S7)

Protective layer-forming coating liquids S2 to S7 were prepared in the same manner as the protective layer-forming coating liquid S1, except that the type of the electron transporting compound and the amount of the curable compound (M-9050) were changed as shown in Table 2, and for S5 to s7, the solvent was also changed to a mixed solvent of THF/2-propanol in a mass ratio of 3/7.

### <Production of Single-Layered Photoconductor>

Single-layered photoconductors were produced by the following procedures.

### Example 6

The undercoat layer-forming coating liquid P1 was immersion-coated on an aluminum cylinder having a diameter of 30 mm and a length of 244 mm with a machined surface to form an undercoat layer having a film thickness of 0.3 μm after drying. The single-layered photosensitive layer-forming coating liquid Q1 was immersion-coated on the undercoat layer and dried at 100° C. for 24 minutes to form a single-layered photosensitive layer having a film thickness of 32 μm after drying. The protective layer-forming coating liquid S1 was ring-coated on the single-layered photosensitive layer, and irradiated with LED light of 365 nm at an intensity of 0.9 W/cm² (108 J/cm²) for 2 minutes while rotating the photoconductor at 60 rpm under a nitrogen atmosphere immediately after the coating to form a protective layer having a film thickness of 1.5 μm after curing, thereby producing a photoconductor A1.

### Examples 7 to 9 and Comparative Examples 5 to 7

Photoconductors A2 to A7 were produced in the same manner as the photoconductor A1, except that the protective layer-forming coating liquid S1 was changed to each of the protective layer-forming coating liquids S2 to S7.

### <Electrical Properties: Evaluation of Residual Potential>

Each of the photoconductors A1 to A7 obtained in Examples and Comparative Examples was mounted on an electrophotographic characteristic evaluation apparatus (described in “Zoku Denshishashin Gijutu no Kiso to Oyo” (Sequel to Fundamentals and Applications of Electrophotographic Technology), edited by The Society of Electrophotography of Japan, Corona Publishing Co., Ltd., pp. 404 to 405), prepared in accordance with the measurement standard of The Society of Electrophotography of Japan, to thereby measure the electrical properties through cycles of charging, exposure, potential measurement, and static elimination as follows.

First, the grid voltage was regulated to charge the photoconductor so as to have an initial surface potential (V0) of +700 V. The photoconductor was then irradiated with exposure light of 1.3 μJ/cm² to measure a residual potential (VL) at 60 milliseconds after irradiation. The exposure light used was monochromatic light of 780 nm extracted from halogen lamp light through an interference filter. The measurement was performed in an environment with a temperature of 25° C. and a relative humidity of 50% (N/N environment).

The results of the residual potential (VL) are shown in Table 2. The smaller the absolute value of the residual potential (VL), the more the charge has been sufficiently transported and the potential has been lowered, which can be considered a good result.

In the present invention, those having a residual potential (VL) of 320 V or less were evaluated as “pass”. In Comparative Examples 5 and 7, the solubility of the electron transporting compound was poor and a uniform protective layer could not be formed, and thus the residual potential could not be measured.

### <Electrical Properties: Evaluation of Dark Decay Rate>

Each of the photoconductors A1 to A7 obtained in Examples and Comparative Examples was mounted on an electrophotographic characteristic evaluation apparatus (described in “Zoku Denshishashin Gijutu no Kiso to Oyo” (Sequel to Fundamentals and Applications of Electrophotographic Technology), edited by The Society of Electrophotography of Japan, Corona Publishing Co., Ltd., pp. 404 to 405), prepared in accordance with the measurement standard of The Society of Electrophotography of Japan, to thereby measure the electrical properties through cycles of charging, exposure, potential measurement, and static elimination as follows.

To evaluate the electrical properties, the dark decay rate (DDR) (%) was measured after charging to +700 V and holding for 5 seconds. The measurement was performed in an environment with a temperature of 25° C. and a relative humidity of 50% (N/N environment).

The results of the dark decay rate (DDR) are shown in Table 2. The dark decay rate (DDR) refers to the retention rate (%) of the surface potential when a photoconductor having a charged surface is left for a certain period of time. The higher the retention rate (%) of the surface potential, the better the potential is retained over time and the better the chargeability, which can be considered a good result.

In the present invention, those having a dark decay rate (DDR) of 65.0% or more were evaluated as “pass”. In Comparative Examples 5 and 7, the solubility of the electron transporting compound was poor and a uniform protective layer could not be formed, and thus the dark decay rate (DDR) could not be measured.

### <Electrical Properties: Overall Evaluation>

Based on the above, in the present invention, those having a residual potential (VL) of 320 V or less and a dark decay rate (DDR) of 65.0% or more were evaluated as “pass” in the overall evaluation.

TABLE 2

| | Protective | | Protective layer | | Electrical properties | |
|---|---|---|---|---|---|---|
| | layer- | | Electron | Curable | | Dark |
| | forming | Photo- | transporting | compound | Residual | decay |
| | coating | con- | compound | (parts | potential | rate |
| | liquid | ductor | (parts by mass) | by mass) | VL (V) | DDR (%) |
| Example 6 | S1 | A1 | Compound 1 (100) | M9050 (50) | 309 | 72.6 |
| Example 7 | S2 | A2 | Compound 2 (100) | M9050 (50) | 246 | 81.5 |
| Example 8 | S3 | A3 | Compound 3 (100) | M9050 (50) | 119 | 68.0 |
| Example 9 | S4 | A4 | Compound 4 (100) | M9050 (50) | 130 | 70.3 |
| Comparative | S5 | A5 | Comparative | M9050 (50) | not | not |
| Example 5 | | | compound 1 (100) | | measurable | measurable |
| Comparative | S6 | A6 | Comparative | M9050 (100) | 205 | 38.4 |
| Example 6 | | | compound 2 (100) | | | |
| Comparative | S7 | A7 | Comparative | M9050 (100) | not | not |
| Example 7 | | | compound 4 (100) | | measurable | measurable |

### <Discussion>

From the results in Table 2, it was confirmed that all of the photoconductors in Examples 6 to 9 were superior in terms of residual potential properties compared to those in Comparative Examples 5 to 7.

The electron transporting compounds used in Examples 6 to 9 were each the compound represented by the formula (1). The compound is an electron transporting compound having a left-right asymmetric structure in which an electron transporting skeleton is centered, with a side chain having a polymerizable functional group on one side, and with a side chain having no polymerizable functional group on the other side.

From the results of the above Examples and Comparative Examples, as well as the results of the tests conducted by the present inventors to date, it is found that, by adding the electron transporting compound having a left-right asymmetric structure in which an electron transporting skeleton is centered, with a side chain having a polymerizable functional group on one side, and with a side chain having no polymerizable functional group on the other side to the protective layer, the photoconductor can have superior electrical properties, especially residual potential properties. This is presumably because the electron transporting compound, since it has a left-right asymmetry and one of the sides is a side chain having no polymerizable functional group, has low crystallinity and good solubility in solvents, especially alcohols, which improves the coatability when forming a protective layer and enables the formation of a uniform protective layer without irregularities, resulting in good electron transportability in the protective layer and good electrical properties of the photoconductor.

### EXPLANATIONS OF LETTERS OR NUMERALS

1 Photoconductor (Electrophotographic Photoconductor)
2 Charging Device (Charging Roller; Charging Section)
3 Exposing Device (Exposing Section)
4 Developing Device (Developing Section)
5 Transferring Device
6 Cleaning Device
7 Fixing Device
41 Developing Tank
42 Agitator
43 Supplying Roller
44 Developing Roller
45 Regulating Member
71 Upper Fixing Member (Pressure Roller)
72 Lower Fixing Member (Fixing Roller)
73 Heating Device
T Toner
P Printing Paper (Paper, Medium)

1. An electrophotographic photoconductor comprising a photosensitive layer and a protective layer in sequence on a conductive support,
wherein the protective layer comprises a polymer of an electron transporting compound represented by the following formula (1),
A-X-B (1)
wherein X represents an electron transporting skeleton; A is represented by the following formula (2), B is represented by the following formula (3), and A and B are different from each other, 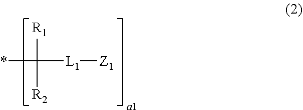
wherein the asterisk (*) represents a bond with the formula (1); R₁ and R₂ each independently represent a hydrogen atom, an optionally substituted alkyl group, an optionally substituted alkoxy group, an optionally substituted aryloxy group, an optionally substituted heteroaryloxy group, an optionally substituted alkoxycarbonyl group, an optionally substituted dialkylamino group, an optionally substituted diarylamino group, an optionally substituted arylalkylamino group, an optionally substituted acyl group, an optionally substituted haloalkyl group, an optionally substituted alkylthio group, an optionally substituted arylthio group, an optionally substituted silyl group, an optionally substituted siloxy group, an optionally substituted aromatic hydrocarbon group, or an optionally substituted aromatic heterocyclic group; L₁ represents a divalent group; Z₁ represents an optionally substituted acryloyl group, an optionally substituted methacryloyl group, an optionally substituted acrylamide group, or an optionally substituted methacrylamide group; a1 is an integer of 1 or more; and when a1 is an integer of 2 or more, each of R₁, R₂, L₁, and Z₁ may be the same or different from each other, 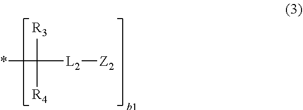
wherein the asterisk (*) represents a bond with the formula (1); R₃ and R₄ each independently represent a hydrogen atom, an optionally substituted alkyl group, an optionally substituted alkoxy group, an optionally substituted aryloxy group, an optionally substituted heteroaryloxy group, an optionally substituted alkoxycarbonyl group, an optionally substituted dialkylamino group, an optionally substituted diarylamino group, an optionally substituted arylalkylamino group, an optionally substituted acyl group, an optionally substituted haloalkyl group, an optionally substituted alkylthio group, an optionally substituted arylthio group, an optionally substituted silyl group, an optionally substituted siloxy group, an optionally substituted aromatic hydrocarbon group, or an optionally substituted aromatic heterocyclic group; L₂ represents a divalent group; Z₂ represents a hydrogen atom, an alkoxy group, or an amide group; b1 is an integer of 1 or more; and when b1 is an integer of 2 or more, each of R₃, R₄, L₂, and Z₂ may be the same or different from each other.

2. The electrophotographic photoconductor according to claim 1, wherein X in the formula (1) comprises at least one selected from the group consisting of the following formulae (A-1) to (A-27), 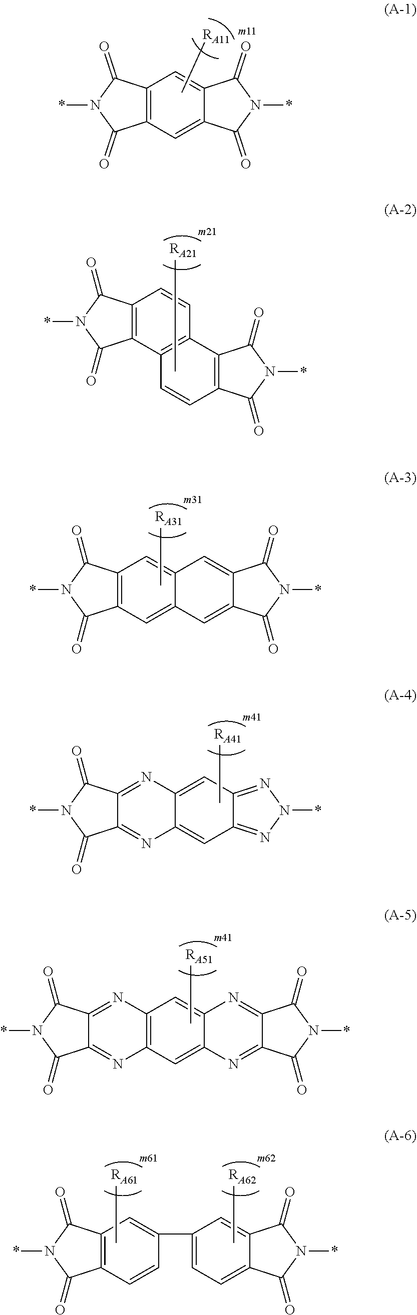 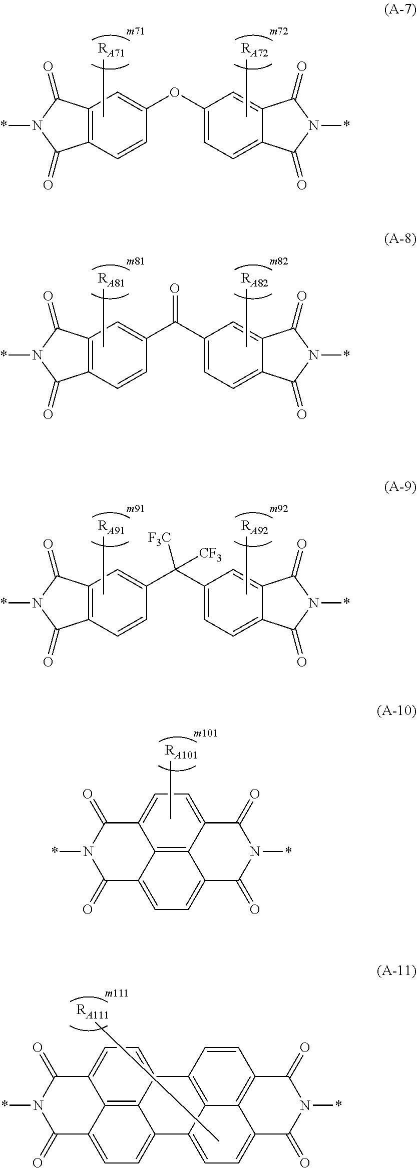 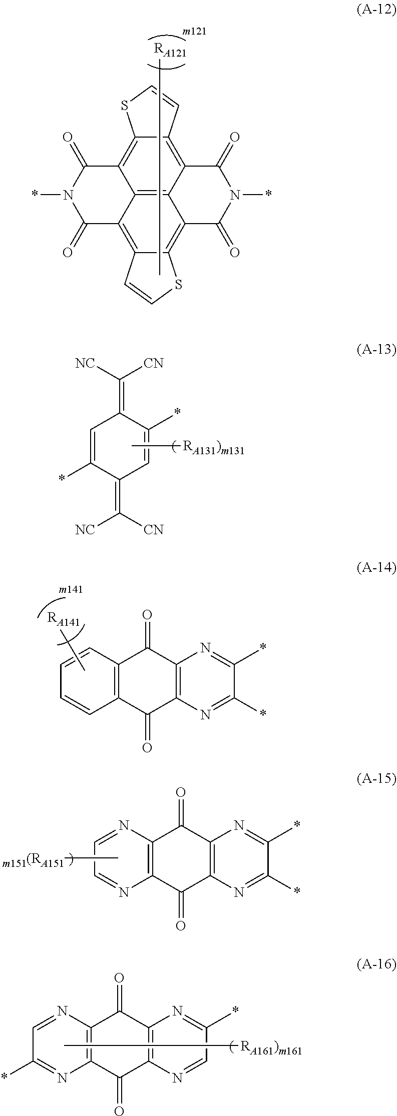 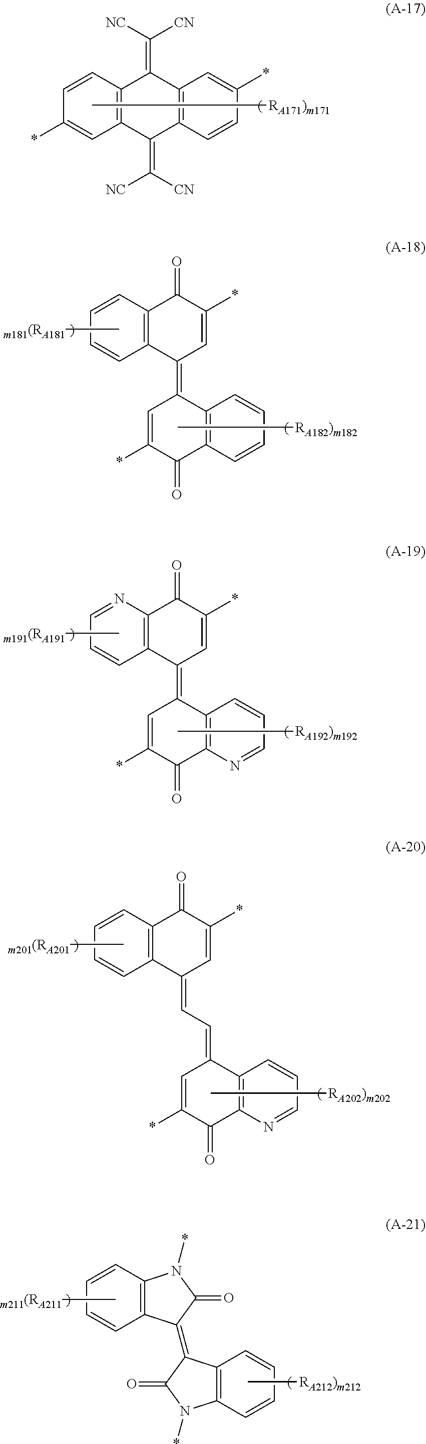 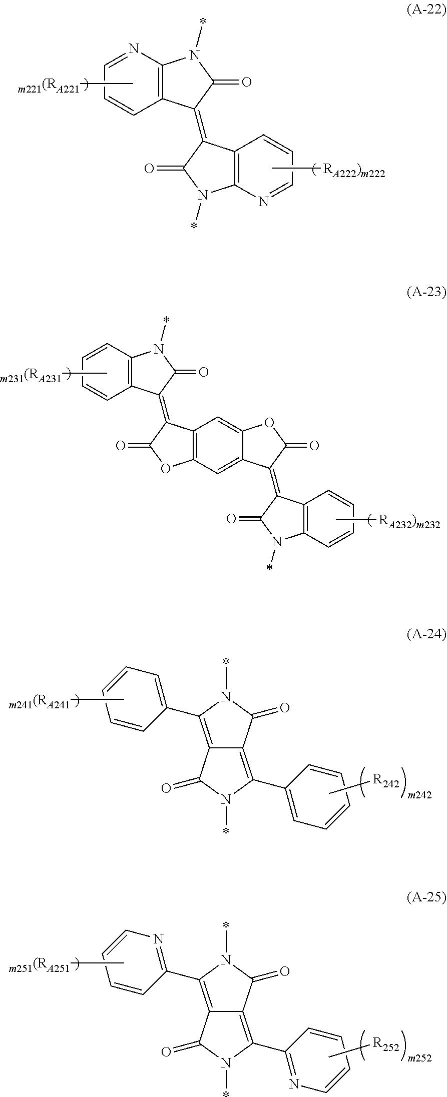 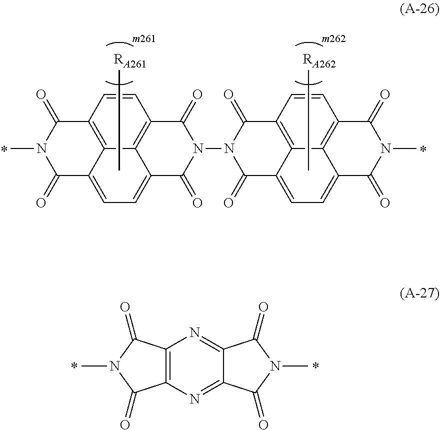
wherein the asterisk (*) represents a bond with the formula (2) or (3); R_{A11}, R_{A21}, R_{A31}, R_{A41}, R_{A51}, R_{A61}, R_{A62}, R_{A71}/R_{A72}, R_{A81}, R_{A82}, R_{A91}, R_{A92}, R_{A101}, R_{A111}, R_{A121}, R_{A131}, R_{A141}, R_{A151}/R_{A161}, R_{A171}, R_{A181}, R_{A182}, R_{A191}, R_{A192}, R_{A201}, R_{A202}, R_{A211}, R_{A212}/R_{A221}, R_{A222}, R_{A231}, R_{A232}, R_{A241}, R_{A242}, R_{A251}, R_{A252}, R_{A261}, and R_{A262} each independently represent a hydrogen atom, an optionally substituted alkyl group, an optionally substituted aralkyl group, an optionally substituted aromatic group, an optionally substituted alkoxy group, an optionally substituted aryloxy group, an optionally substituted acyl group, an optionally substituted ester group, an optionally substituted cyano group, an optionally substituted nitro group, an optionally substituted sulfone group, an optionally substituted hydroxy group, an optionally substituted aldehyde group, or a halogen atom; m11, m21, m31, m41, m51, m61, m62, m71, m72, m81, m82, m91, m92, m191, m192, m201, m202, m211, m212, m221, m222, m231, m232, m241, m242, m251, m252, m261, and m262 each independently represent an integer of 0 or more; and when m11, m21, m31, m41, m51, m61, m62, m71, m72, m81, m82, m91, m92, m101, m111, m121, m131, m141, m151, m161, m171, m181, m182, m191, m192, m201, m202, m211, m212, m221, m222, m231, m232, m241, m242, m251, m252, m261, and m262 are each 2 or more, each of R_{A11} to R_{A262} may be different from each other.

3. The electrophotographic photoconductor according to claim 1, wherein in the formulae (2) and (3), L₁ and L₂ each independently represent an alkylene group, a divalent group having a ketone group, a divalent group having an ether bond, a divalent group having an ester bond, or a group formed by linking these groups.

4. The electrophotographic photoconductor according to claim 1, wherein in the formulae (2) and (3), when R₁ to R₄ are each other than a hydrogen atom, the substituent that R₁ to R₄ may have comprises an alkyl group.

5. The electrophotographic photoconductor according to claim 1, wherein the electron transporting compound represented by the formula (1) comprises at least two or more polymerizable functional groups.

6. An electrophotographic photoconductor cartridge comprising the electrophotographic photoconductor according to claim 1.

7. An image formation device comprising the electrophotographic photoconductor according to claim 1.

8. A compound being represented by the following formula (1),
A-X-B (1)
wherein X represents an electron transporting skeleton; A is represented by the following formula (2), B is represented by the following formula (3), and A and B are different from each other, 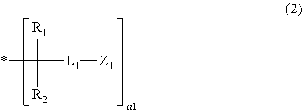
wherein the asterisk (*) represents a bond with the formula (1); R₁ and R₂ each independently represent a hydrogen atom, an optionally substituted alkyl group, an optionally substituted alkoxy group, an optionally substituted aryloxy group, an optionally substituted heteroaryloxy group, an optionally substituted alkoxycarbonyl group, an optionally substituted dialkylamino group, an optionally substituted diarylamino group, an optionally substituted arylalkylamino group, an optionally substituted acyl group, an optionally substituted haloalkyl group, an optionally substituted alkylthio group, an optionally substituted arylthio group, an optionally substituted silyl group, an optionally substituted siloxy group, an optionally substituted aromatic hydrocarbon group, or an optionally substituted aromatic heterocyclic group; L₁ represents a divalent group; Z₁ represents an optionally substituted acryloyl group, an optionally substituted methacryloyl group, an optionally substituted acrylamide group, or an optionally substituted methacrylamide group; a1 is an integer of 1 or more; and when a1 is an integer of 2 or more, each of R₁, R₂, L₁, and Z₁ may be the same or different from each other, 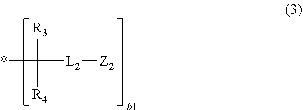
wherein the asterisk (*) represents a bond with the formula (1); R₃ and R₄ each independently represent a hydrogen atom, an optionally substituted alkyl group, an optionally substituted alkoxy group, an optionally substituted aryloxy group, an optionally substituted heteroaryloxy group, an optionally substituted alkoxycarbonyl group, an optionally substituted dialkylamino group, an optionally substituted diarylamino group, an optionally substituted arylalkylamino group, an optionally substituted acyl group, an optionally substituted haloalkyl group, an optionally substituted alkylthio group, an optionally substituted arylthio group, an optionally substituted silyl group, an optionally substituted siloxy group, an optionally substituted aromatic hydrocarbon group, or an optionally substituted aromatic heterocyclic group; L₂ represents a divalent group; Z₂ represents a hydrogen atom, an alkoxy group, or an amide group; b1 is an integer of 1 or more; and when b1 is an integer of 2 or more, each of R₃, R₄, L₂, and Z₂ may be the same or different from each other.

9. The compound according to claim 8, wherein X in the formula (1) comprises at least one selected from the group consisting of the following formulae (A-1) to (A-27), 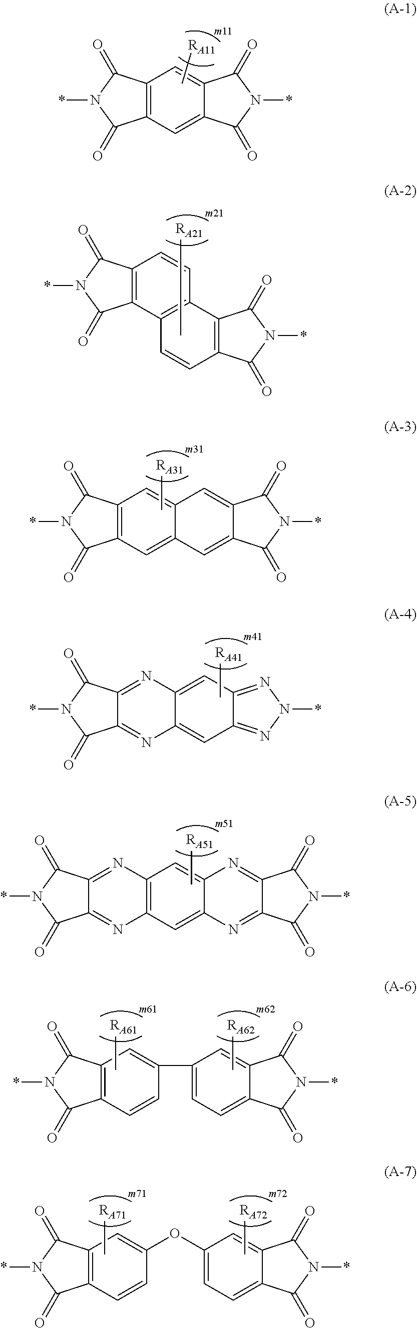 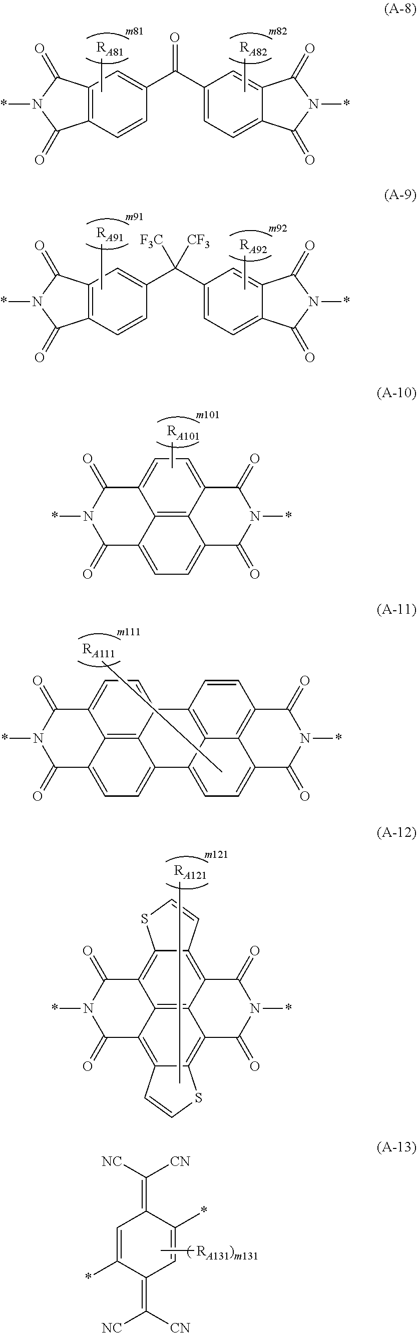 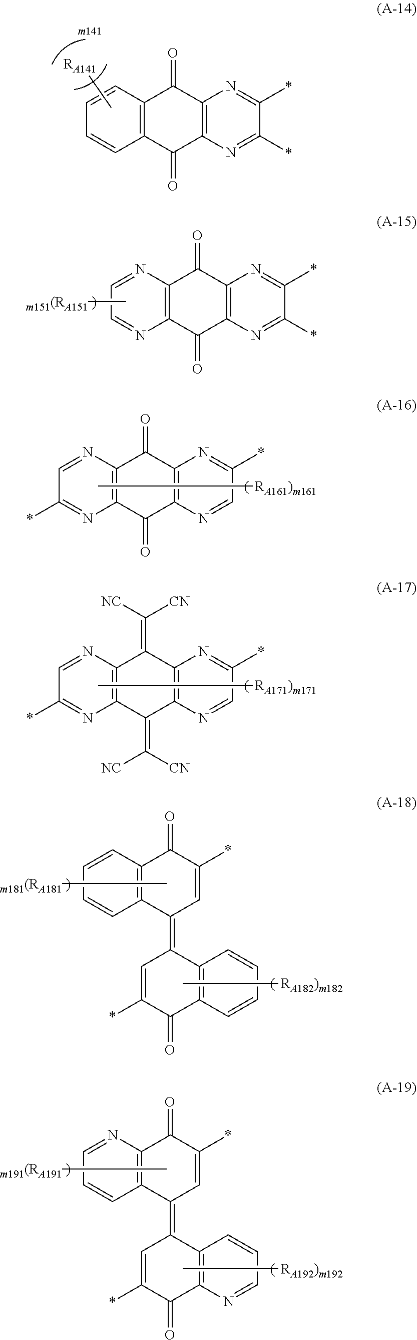 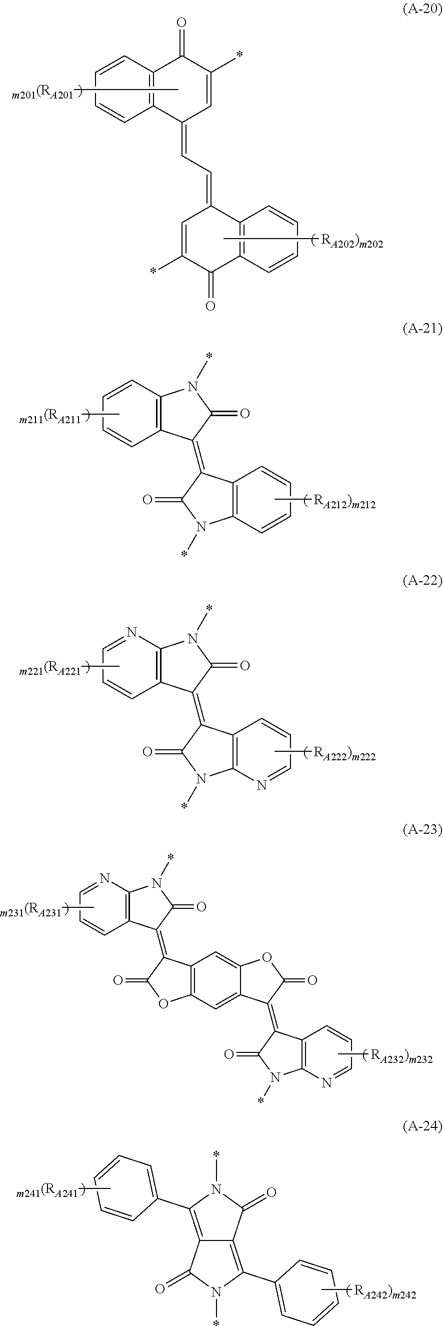 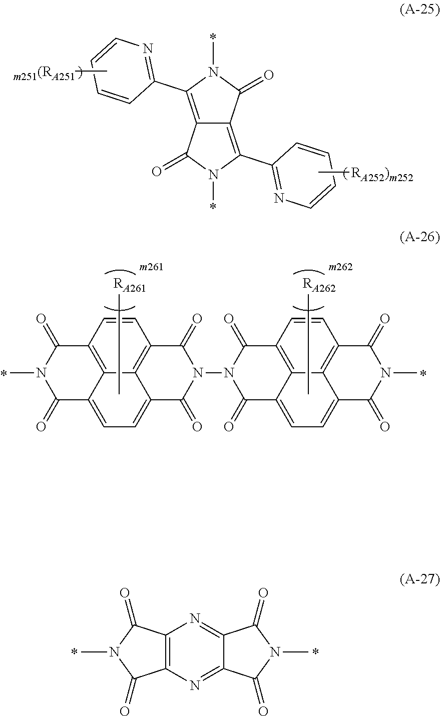
wherein the asterisk (*) represents a bond with the formula (2) or (3); R_{A11}, R_{A21}, R_{A31}, R_{A41}, R_{A51}, R_{A61}, R_{A62}, R_{A71}/R_{A72}, R_{A81}, R_{A82}, R_{A91}, R_{A92}, R_{A101}, R_{A111}, R_{A121}, R_{A131}, R_{A141}, R_{A151}/R_{A161}, R_{A171}, R_{A181}, R_{A182}, R_{A191}, R_{A192}, R_{A201}, R_{A202}, R_{A211}, R_{A212}/R_{A221}, R_{A222}, R_{A231}, R_{A232}, R_{A241}, R_{A242}, R_{A251}, R_{A252}, R_{A261}, and R_{A262} each independently represent a hydrogen atom, an optionally substituted alkyl group, an optionally substituted aralkyl group, an optionally substituted aromatic group, an optionally substituted alkoxy group, an optionally substituted aryloxy group, an optionally substituted acyl group, an optionally substituted ester group, an optionally substituted cyano group, an optionally substituted nitro group, an optionally substituted sulfone group, an optionally substituted hydroxy group, an optionally substituted aldehyde group, or a halogen atom; m11, m21, m31, m41, m51, m61, m62, m71, m72, m81, m82, m91, m92, m191, m192, m201, m202, m211, m212, m221, m222, m231, m232, m241, m242, m251, m252, m261, and m262 each independently represent an integer of 0 or more; and when m11, m21, m31, m41, m51, m61, m62, m71, m72, m81, m82, m91, m92, m101, m111, m121, m131, m141, m151, m161, m171, m181, m182, m191, m192, m201, m202, m211, m212, m221, m222, m231, m232, m241, m242, m251, m252, m261, and m262 are each 2 or more, each of R_{A11} to R_{A262} may be different from each other.

10. The compound according to claim 8, wherein in the formulae (2) and (3), L₁ and L₂ each independently represent an alkylene group, a divalent group having a ketone group, a divalent group having an ether bond, a divalent group having an ester bond, or a group formed by linking these groups.

11. The compound according to claim 8, wherein in the formulae (2) and (3), when R₁ to R₄ are each other than a hydrogen atom, the substituent that R₁ to R₄ may have comprises an alkyl group.

12. The compound according to claim 8, comprising at least two or more polymerizable functional groups.
